(19) United States
(12) Patent Application Publication
(10) Pub. No.: US 2015/0203556 A1
(43) Pub. Date: Jul. 23, 2015

(54) COMPOSITIONS AND METHODS FOR MODULATION OF IL-20 FAMILY CYTOKINE ACTIVITY

(71) Applicant: The UAB Research Foundation, Birmingham, AL (US)

(72) Inventor: Mark Walter, Birmingham, AL (US)

(21) Appl. No.: 14/413,756

(22) PCT Filed: Jul. 10, 2013
(86) PCT No.: PCT/US2013/049865

§ 371 Date: Jan. 9, 2015

Related U.S. Application Data
(60) Provisional application No. 61/669,935, filed on Jul. 10, 2012.

(51) Int. Cl. C07K 14/54; G01N 33/68

(52) U.S. Cl. CPC C07K 14/54; G01N 33/6869; G01N 2500/02

(57) ABSTRACT

Provided herein are compositions comprising one or more agents that inhibit or reduce the activity of IL-20. Optionally, the agents further inhibit or reduce the activity of IL-24. Kits and method of treating or preventing an inflammatory or autoimmune disease using the compositions are also provided. In addition, provided are methods of screening for agents that selectively bind the IL20R1/IL-20R2 receptor complex or that selectively bind the IL-22R1/IL-20R2 receptor complex. The methods include contacting an agent to be screened with the receptor and determining whether the agent selectively binds to the receptor.

This application claims the benefit of U.S. Provisional Application No. 61/669,935, filed Jul. 10, 2012, which is hereby incorporated herein in its entirety.

### STATEMENT REGARDING FEDERALLY FUNDED RESEARCH

This invention was made under government support under Grant Nos. AI49342, AI47300, and R21AI081065 awarded by the National Institutes of Health. The government has certain rights in this invention.

### BACKGROUND

Interleukin-20 (IL-20) is an α-helical cytokine discovered by EST database mining and mapped to human chromosome 1q32 in a cluster with IL-10, IL-19, and IL-24 (MDA7). Based on genomic location, amino acid sequence identity (32%-40%), and use of a common receptor chain for signaling (IL-20R2), IL-19, IL-20, and IL-24 have been designated as IL-20 subfamily cytokines (SFCs). The IL-20 SFCs, IL-10, IL-22 and IL-26 form the IL-10 cytokine family, which together with the interferons (Type I, IFN-α/β; Type II, IFN-γ; and type III, IFN-λs or IL-28/IL-29) form the class 2 cytokine family.

IL-20 SFCs induce cellular signaling through a common receptor heterodimer composed of IL-20R1 and IL-20R2 chains (the type I complex). IL-20 and IL-24 also signal through an IL-22R1/IL-20R2 heterodimer (type II complex), whereas IL-19 only signals through the type I complex. IL-20R1 and IL-22R1 also pair with the IL-10R2 chain to form receptor heterodimers that induce cell signaling upon IL-26 (IL-20R1/IL-10R2) and IL-22 (IL-22R1/IL-10R2) binding, respectively. Despite promiscuous pairing of the R2 chains, IL-20R2 cannot substitute for IL-10R2 in IL-22 signaling. Furthermore, IL-19, IL-20, and IL-24 appear to have largely non-redundant biological activities in vivo, suggesting they may engage the type I and type II receptor heterodimers differently. However, a mechanistic basis for such differences has not been determined.

IL-20 has been implicated in the pathophysiology of psoriasis. Transgenic mice over expressing IL-20 exhibit a phenotype similar to human psoriatic skin, and IL-20 neutralizing antibodies resolve psoriasis in a human xenograft transplantation model. Increased levels of IL-19 and IL-24 are also observed in skin samples from psoriasis patients, but the significance and/or biological function of IL-19 and IL-24 is unclear. IL-24 transgenic (Tg) mice exhibit epidermal hyperplasia and proliferation, suggesting IL-24 activity in vivo is similar to IL-20. However, IL-19 Tg mice were reported to exhibit a normal skin phenotype, which is consistent with IL-19's unique receptor specificity. IL-20 is also implicated in rheumatoid arthritis (RA) and atherosclerosis. IL-20 also exhibits arteriogenic/angiogenic properties and may be important for treating ischemic disease. IL-20 was found to induce osteoclastogenesis, by up-regulating the RANK-RANKL signaling proteins and may be a therapeutic target for osteoporosis.

Expression of the IL-20R1/IL-20R2 and IL-22R1/IL-20R2 heterodimers that are required for IL-20 biological activity, have only been observed on cells of epithelial origin including skin, lung, and testis. These data suggest a major role for IL-20 SFCs in mediating cross-talk between infiltrating immune cells (T-cells, M0, and DC) that express the IL-20 SFCs, and the skin. However, in some cases the pleotropic activities of IL-20 are at odds with the cellular expression of the IL-20 receptors. In particular, IL-20 and IL-19 induce naïve T-cells toward a TH2 secretory phenotype, characterized by increased IL-4, IL-13, and reduced IFNγ production. However, IL-20R2, but not IL-20R1 or IL-22R1, have been detected in immune cells. In addition, IL-20R2 knockout mice exhibit disrupted CD4+ and CD8+T-cell function, which implicates the IL-20 SFCs in T-cell signaling, despite the absence of the IL-20R1 and IL-22R1 receptor chains on these cells.

IL-20 has emerged as a highly pleotropic cytokine involved in essential cellular processes and pathology. Despite advances and an improved understanding of IL-20 biology, the molecular mechanisms that allow IL-20, IL-19, and IL-24 to discriminate and activate the type I (IL-20R1/IL-20R2) and type II (IL-22R1/IL-20R2) receptor complexes are unknown.

### SUMMARY

Provided herein are compositions comprising one or more agents that inhibit or reduce the activity of IL-20. Optionally, the agents further inhibit or reduce the activity of IL-24. Kits and methods of treating or preventing an inflammatory or autoimmune disease using the compositions are also provided.

Provided are also methods of screening for agents that selectively bind the IL-20R1/IL-20R2 receptor complex or that selectively bind the IL-22R1/IL-20R2 receptor complex. The methods include contacting an agent to be screened with the receptor and determining whether the agent binds to the receptor.

The details of one or more embodiments are set forth in the accompanying drawings and the description below. Other features, objects, and advantages will be apparent from the description and drawings, and from the claims.

### DESCRIPTION OF DRAWINGS

FIG. 1 is a schematic diagram of IL-10 family receptor complexes. The box distinguishes IL-20R2 signaling complexes from IL-10R2 complexes, which share identical R1 chains.

FIGS. 2A-2D are diagrams showing the IL-20/IL-20R1/IL-20R2 ternary complex. FIG. 2A is a ribbon diagram of the IL-20 ternary signaling complex. FIG. 2B is a ribbon diagram of IL-19 and IL-20. FIG. 2C is a superposition of the R1 chains, IL-10R1, IL-20R1, and IL-22R1, on the left and R2 chains, IL-10R2 and IL-20R2 on the right. FIG. 2D is a diagram showing the IL-20 dimeric complex observed in the asymmetric unit of the crystals.

FIGS. 3A-3D are graphs showing analysis of IL-19, IL20, IL-22 binding to IL-22R1/IL-20R2-FCkh and IL-22 binding to IL-22R1. FIG. 3A is a graph of the surface Plasmon resonance (SPR) sensorgram data of IL-22 binding to IL-22R1ddq-FCkh. FIG. 3B is a graph of the surface Plasmon resonance (SPR) sensorgram data of IL-22 binding to IL-22R1ddq/IL-20R2-FCkh. FIG. 3C is a graph of the surface Plasmon resonance (SPR) sensorgram data of IL-19 binding to IL-22R1ddq/IL-20R2-FCkh. FIG. 3D is a graph of the surface Plasmon resonance (SPR) sensorgram data of I1-20 binding to IL-22R1 ddq/IL-20R2-FCkh.

FIGS. 4A-4D are schematic diagrams of soluble receptor heterodimers. FIG. 4A is a diagram of the IL-22R1/IL-20R2-FCkh receptor. FIG. 4B is a diagram of IL-22R1-FCkh (MONO) receptor. FIG. 4C is a diagram of the IL-20R1-22R1 hybrid/IL-20R2-FCkh molecule, and FIG. 4D is a diagram of the IL-22R1-20R1hybrid/IL-20R2-FCkh molecule.

FIGS. 5A-5D show the structure and sequence of IL-19 and IL-20. FIG. 5A is a ribbon diagram of IL-19. FIG. 5B is a ribbon diagram of the superposition of IL-19 and IL-20. FIG. 5C is a ribbon diagram of IL-20. FIG. 5D shows the sequence alignment of IL-19 (SEQ ID NO:1), IL-20 (SEQ ID NO:3), and IL-24 (SEQ ID NO:9). The structural variations at the N-/C-termini provide the structural basis for preventing IL-19 from binding to IL-22R1/IL-20R2 type-II complex.

FIGS. 6A-6D are diagrams of the structural comparison of IL-10 family receptors. FIGS. 6A and 6B are orthogonal views of the IL-10 family receptors, IL-10R1, IL-20R1, IL-22R1, IL-10R2, and IL-20R2, with their D1 domains superimpose sed. The box in FIG. 6A highlights the D1 L2 loops, enlarged in panels C and D, which present essential tyrosine residues to bind ligand. The comparisons highlight 1) the unique inter-domain angle of IL-20R2, relative to the other receptors; 2) contrasts the highly conserved YG motif in the L2 loops of the R1 chains IL-10R1, IL-20R1, and IL-22R1, compared to diverse β-hairpin L2 loop of IL-10R2 and the α-helix in the L2 loop of IL-20R2; and 3) the L2 loops of the R1 and R2 chains exhibit a handedness, where L2 loop tyrosines of the R1 chains resemble fingers of a left hand, and the R2 chain tyrosines function as a right hand to capture cytokines between them. Tyrosine residues shown are Tyr-43^{IL-10R1}, Tyr-76^{IL-20R1}, Tyr-60^{IL-22R1}, Tyr-40^{IL-10R2}, and Tyr-74^{IL-20R2}/Tyr-78^{IL-20R2}.

FIGS. 7A-7F are diagrams and sequences showing the structure, affinity and specificity of the site 1 interface shown in FIG. 2A. FIG. 7A is a ribbon diagram of the IL-20/IL-20R1 interface. FIG. 7B is a diagram of the superposition of IL-10/IL-10R1, IL-20/IL-20R1 and IL-22/IL-22R1 binary complexes. FIG. 7C is a diagram of the comparison of site 1a between IL-10/IL-10R1 and IL-20/IL-20R1. Dashed lines in the circle are IL-10-specific hydrogen bonds. Black dashed lines correspond to IL-20/IL-20R1 hydrogen bonds. FIG. 7D is a ribbon diagram of the comparison of site 1a between IL-22/IL-22R1 and IL-20/IL-20R1. Dashed lines in the circle are IL-22-specific hydrogen bonds. Black dashed lines correspond to hydrogen bonds conserved in IL-20 and IL-22 interfaces. FIGS. 7E and 7F are diagrams showing site 1b steric clashes observed for non-cognate complexes. See FIG. 1. IL-19 (E) clashes with IL-22R1 L6 loop, but not IL-20R1 L6. IL-22 (F) clashes with the IL-20R1 L6 loop, but not the IL-22R1 L6 loop.

FIGS. 8A-8E are diagrams showing the IL-20 site 2 interface and mechanisms controlling affinity and specificity. FIG. 8A is a ribbon diagram of the IL-20/IL-20R2 interface. FIG. 8B is a ribbon diagram showing detailed contacts in the IL-20/IL-20R2 site 2 interface. FIG. 8C is a ribbon diagram showing the conformational differences in helix C between IL-20 bound to IL-20R2 and unbound IL-19. The IL-20R2 L2 loop is also shown in the figure. FIG. 8D is a ribbon diagram showing the contacts between IL-19 and IL-20R2, not found in IL-20/IL-20R2, that modulate increased IL-19/IL-20R2 affinity. FIG. 8E is a ribbon diagram showing the specificity mechanism preventing non-cognate IL-22 from binding to IL-20R2 is controlled by IL-22 helix D residue, Phe-105^{IL-22}. Cognate IL-20 is also shown in the figure (also see FIG. 1).

FIGS. 9A-9D show the IL-20 site 3 interface is essential and distinct from other complexes. FIG. 9A is a ribbon diagram showing the site 3 interface between IL-20R1 and IL-20R2. FIG. 9B is a graph showing the importance of the site 3 interface in ternary complex formation. SPR sensograms obtained by injecting cytokines (IL-19 or IL-20, at 250 nM) and/or receptors (IL-20R1 or M3, at 425 nM), as labeled on the figure, over an IL-20R2 coupled biacore chip. Injection of IL-20+IL-20R1 results in a large response consistent with ternary complex formation. However, injection of IL-20+M3, an IL-20R1 site 3 mutant with S190^{IL-20R1}, W194^{IL-20R1}, and W207^{IL-20R1} mutated to alanine drastically reduces binding due to disruption of ternary complex formation. Similar results are observed for complex formation by IL-19. FIG. 9C is a ribbon diagram of the superposition of the D2 domains of GHR site 1, IL-6R, and IL-20R1. The positions of the D2 domains of GHR site2, GP130, and IL-20R2 are shown, along with a schematic of the differences in their orientations. FIG. 9D is a ribbon diagram showing the putative model of the IL-22/IL-22R1/IL-10R2 complex based on the IL-20 ternary complex structure. The D2 domains of the model are separated by 15 [symbol] suggesting IL-10R2 complexes are distinct from IL-20R2 complexes.

FIGS. 10A-10E are ribbon diagrams showing the IL-20/IL-20R1/IL-20R2 dimer complex observed in the crystals. FIG. 10A is a ribbon diagram showing the protein interfaces in the IL-20/IL-20R2 dimer. Each IL-20R2 forms two contacts. The site 2 interface and an IL-20/IL-20R2-D2 interface. FIGS. 10B and 10C are ribbon diagrams showing the IL-20/IL-20R2 dimer in parallel (FIG. 10B) and perpendicular (FIG. 10C) to the two-fold axis. The putative location of the cell membrane is also shown. FIGS. 10D and 10E show identical views of the complex that include the IL-20R1 chains.

FIGS. 11A-11C are pictures of gels showing the cross-linking of IL-19 and IL-20 Receptor Complexes. FIG. 11A shows IL-19 is unable to assemble a 2:2 IL-19/IL-20R2 complex. FIGS. 11B and 11C show weak bands corresponding to dimeric ternary complexes for IL-19 and IL-20 ternary complexes. The molecular weights of the proteins used in the experiments are IL-19 (19,500 Da), IL-20 (17,600 Da), IL-20R2 (23,000 Da), IL-20R1 (30,000 Da). (Da=daltons.) CTL lanes correspond to the proteins without cross-linking agent. Lanes 1, 2, 3, and 4 correspond to 30 minute cross-linking experiments with 0.005%, 0.01%, 0.02%, and 0.05% glutaraldehyde, respectively.

FIGS. 12A-12H are graphs showing IL19/IL-20 receptor interactions and complex stability. SPR sensorgrams, data fits (black lines), KD values, and residual errors obtained for complexes described at the top of each panel. Panels are labeled as injected soluble proteins, followed by a semicolon (:), then the protein coupled to chip surface, IL-20R1 or IL-20R2. FIGS. 12A and 12B show injection of IL-19 and IL-20 (max conc.=10 μM, 2 fold diluted to 0.625 μM) over IL-20R1. FIGS. 12C and 12D show injection of IL-19 (max conc.=500 nM, 2-fold diluted to 3.91 nM) or IL-20 (max conc.=2000 nM, 2-fold diluted to 62.5 nM) over IL-20R2. FIGS. 12E-12H show IL-19 and IL-20 (fixed at 250 nM) mixed with different concentrations of IL-20R1, or IL-22R1, (max conc.=1000 nM, 2-fold diluted to 7.81 nM) and injected over IL-20R2. To estimate R1 chain affinity/complex stability, the contribution of the ligand was removed by using the 250 nM IL-19, or IL-20, sensorgram as the blank subtraction. All sensorgrams were fit to 1:1 binding models. Kinetic parameters are listed in Table 4.

FIGS. 13A-13D are graphs showing stability of IL-19 and IL-20 ternary complexes. Stability of the ternary complexes were determined by co-injecting IL-19+soluble IL-20R2 (FIGS. 13B and 13D), or IL-20+soluble IL-20R2 (FIGS. 13A and 13C), over an IL-20R1-FC coupled surface. The concentration of soluble IL-20R2 in these experiments was fixed at 500 nM. IL-19 and IL-20 concentrations varied from 1000 nM to 31.25 nM (2 fold dilutions). IL-19/soluble IL-20R2 and IL-20/IL-20R2 were also injected over an IL-22R1 coupled surface. The SPR sensorgrams are labeled as injected proteins, followed by a semicolon (:), then the protein coupled to the chip surface, IL-20R1 or IL-22R1. All sensograms yielding binding responses were fit to 1:1 binding models.

FIGS. 14A-14D are ribbon diagrams showing IL-20R2 and IL-10R2 complexes are distinct. FIG. 14A shows the R1 chain complexes are easily superimposed. Specifically, FIG. 14A shows superposition of IL-10/IL10R1 and IL-22/IL-22R1 binary complexes onto the IL-20/IL-20R1/IL-20R2 ternary complex. FIG. 14B shows the D2 domain of the IL-10R2 chain superimposed onto IL-20R2 D2. FIG. 14C shows the D1 domain of IL-10R2 superimposed onto the IL-20R2 D1 domain. FIG. 14D shows orthogonal views of the averaged DAMIN bead models derived from SAXS experiments on IL-10R2. The black line in the unoccupied region of the bead models is 57 [symbol] long and corresponds to the 16-residue N-terminal His-6 tag, which in its extended form (3.5 [symbol] spacing) would be ˜56 [symbol] long. The far right image shows the distinct inter-domain angles of IL-10R2 and IL-20R2 and confirms the D1/D2 inter-domain angle of IL-10R2 in solution is the same as in the crystal structure.

### DETAILED DESCRIPTION

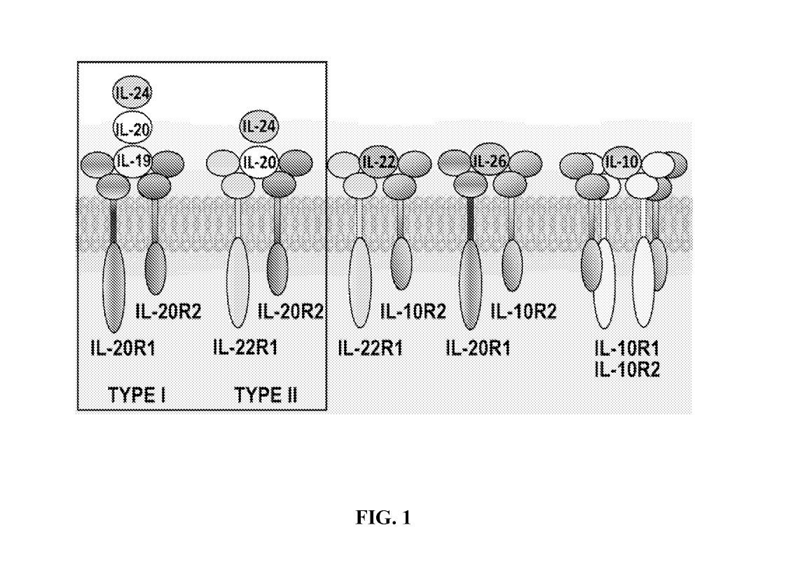

Increased levels of IL-20 may contribute to psoriasis and rheumatoid arthritis. Due to cytokine sharing between the receptors (FIG. 1), it has previously not been possible to target either complex specifically with a neutralizing antibody to a cytokine or one of the receptor chains. The structural and biochemical studies described herein provide methods for IL-20 neutralization and also for generation of antagonists that block IL-20 as well as IL-24, which show the same receptor specificity (FIG. 1) and similar biological activities. Exemplary specific molecules designed based on the structural and biochemical studies and described herein include a soluble IL-22R1/IL20R1 hybrid receptor plus IL20R2 receptor—FC heterodimer to selectively neutralize IL-20 and IL-24 biological activity; an IL-20R1/IL-22R1 hybrid receptor plus IL-20R2 receptor—FC heterodimer to selectively neutralize I1-20 and IL-24 biological activity; IL-20 mutant and hybrid proteins that selectively and specifically binds to the IL-20R1/IL-20R2 complex; and IL-22 mutant proteins that selectively binds to the IL-22R1/IL-20R2 complex.

Provided herein are compositions comprising an agent that inhibits or reduces the activity of IL-20. Optionally, the agent selectively binds an IL-20R1/IL-20R2 receptor complex. Optionally, the agent prevents or reduces IL-20 binding in the IL-20R1/IL-20R2 complex. Optionally, the agent does not bind an uncomplexed receptor, e.g., the agent does not bind either IL-20R1 or IL-20R2 alone. By way of example, an agent that selectively binds an IL-20R1/IL-20R2 receptor complex comprises IL-20 (SEQ ID NO:3) comprising one or more mutations. Optionally, the one or more mutations are located in the N-terminus of IL-20. Optionally, the mutation is a mutation in the glutamine at position 40 of SEQ ID NO:3. Optionally, the mutated IL-20 comprises SEQ ID NO:19 or SEQ ID NO:20.

Optionally, the agent that selectively binds an IL-20R1/IL-20R2 receptor is an IL-19/IL-20 hybrid molecule or protein. Optionally, the agent comprises SEQ ID NO:21.

Agents that selectively bind the IL-20R1/IL-20R2 receptor complex and inhibit or reduce the activity of IL-20 include agents that contact one or more amino acids or IL-20R1 and/or IL-20R2. For example, the agent, optionally, contacts amino acids Gly-224, Pro-225 and Pro-226 of IL-20R1. Optionally, the agent contacts one or more of the amino acids selected from the group consisting of Gly-224, Arg-128, Gly-77, Tyr-76, Glu-105, Gln-107, Pro-225, and Pro-226, of IL-20R1. Optionally, the agent contacts one or more of the amino acids selected from the group consisting of Glu-164, Arg-133, Tyr-74, Glu-75, and Thr-104, of IL-20R2. As used herein, individual amino acid residues are considered to be in contact with an agent if any atom (e.g., a heavy or non-hydrogen atom) of the residue is in contact (e.g., Van der Waal's contact) with an atom of the agent. By way of example, interatomic contact can be determined using surface-based algorithms, such as ligand-protein contacts (LPC) (Sobolev et al., Bioinformatics 15(4):327-32 (1999)).

Agents that inhibit or reduce the activity of IL-20 also include agents that selectively bind an IL-22R1/IL-20R2 receptor complex. Such agents can, for example, prevent or reduce IL-20 binding to the IL-22R1/IL-20R2 complex. Agents that selectively bind an IL-22R1/IL-20R2 complex include, but are not limited to, agents comprising an IL-22 (SEQ ID NO:23) with one or more mutations. Optionally, the mutation is located in the CD loop region of IL-22. Optionally, the mutation is a mutation in the tyrosine at position 13 of SEQ ID NO:41. Optionally, the mutation is a mutation from tyrosine to alanine Optionally, the mutation is a mutation in the phenylalanine at position 105 of SEQ ID NO:23. Optionally, the mutated IL-22 does not bind IL-10R2. Optionally, the mutated IL-22 comprises SEQ ID NO:25.

Agents that inhibit or reduce the activity of IL-20, optionally, further inhibit or reduce the activity of IL-24. Optionally, the agent prevents or reduces IL-24 binding to the IL-22R1/IL-20R2 receptor complex and/or the IL-20R1/IL-20R2 complex.

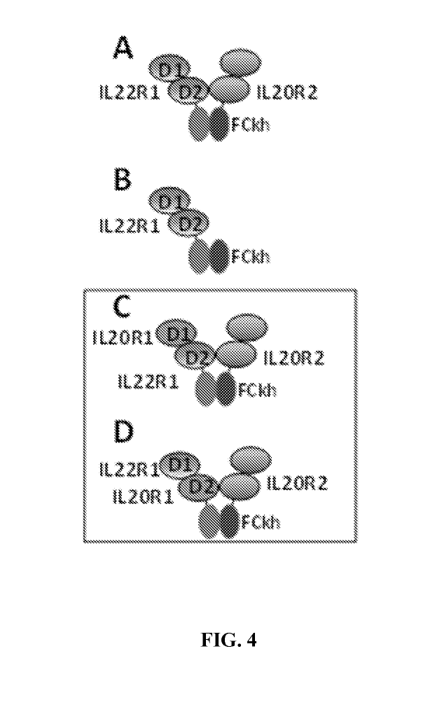

Also provided herein are agents that inhibit or reduce the activity of IL-20 and/or IL-24 by selectively binding IL-20 and IL-24. Such agents include, for example, IL-22R1/IL-20R1 hybrid molecules and IL-20R1/IL-22R1 hybrid molecules. Optionally, the IL-22R1/IL-20R1 hybrid comprises the D1 domain of IL-22R1 and the D2 domain of IL-20R1. Optionally, the IL-22R1/IL-20R1 hybrid comprises SEQ ID NO:27. Optionally, the IL-20R1/IL-22R1 hybrid comprises the D1 domain of IL-20R1 and the D2 domain of IL-22R1. Optionally, the IL-20R1/IL-22R1 comprises SEQ ID NO:29. Optionally, the IL-22R1/IL-20R1 and IL-20R1/IL-22R1 hybrids are expressed as modified fusion proteins, e.g., SEQ ID NOs: 31 and 33. Compositions comprising the IL-20R1/IL-22R1 or IL-22R1/IL-20R1 hybrid molecules can further comprise an IL-20R2 receptor-FC heterodimer. Optionally, the IL-20R2 receptor-FC heterodimer comprises SEQ ID NOs: 7 or 15. Optionally, receptor complexes with an IL-20R1/IL-22R1 hybrid or an IL-22R1/IL-20R1 hybrid and IL-20R2 are in soluble form. This is achieved by linking the hybrid proteins and the IL-20R2 molecules to FC, e.g., FCk or FCh, domains. See FIG. 4. Optionally, the membrane spanning domain and the intracellular domain of the receptors is removed. The receptor complex heterodimers are formed through interactions in the FCk and FCh domains fused to the receptor hybrids and IL-20R2. See FIGS. 4C and 4D. Exemplary FC sequences include, but are not limited to FCk (amino acids 222-449 of SEQ ID NO:31) and FCh (amino acids 217-460 of SEQ ID NO:33) from murine IgG-2a. Under normal biological conditions, the IL-20R1/IL-20R2 and IL-22R1/IL-20R1 receptor complexes are located in cellular membranes and, thus, comprise a transmembrane or hydrophobic region inhibiting the generation of these receptors in a soluble form. In contrast, the IL-20R1/IL-22R1-FC and IL-20R2-FC receptor complexes and the IL-22R1/IL-20R1-FC and IL-20R2-FC receptor complexes provided herein are in soluble form advantageously allowing their use in laboratory and/or clinical settings.

Optionally, the provided polypeptides may comprise other sequences including, purification tags and cleavage sites for removing the purification tags. Such purification tags and cleavage sites can be located on the N-terminus or C-terminus of the polypeptide. Suitable purification tags and cleavage sites include, but are not limited to, SEQ ID NO:42 (IEGRLESRGPFEGKPIPNPLLGLDSTRTGHHHHHH); SEQ ID NO:43 (IEGRVESRGPFEGKPIPNPLLGLDSTRTGHHHHHH; and SEQ ID NO:44 (HHHHHHSDIEGRA).

As used herein, the binding of an agent to a receptor complex is understood to be selective. By selectively binds, selectively binding, or specifically binding refers to the agent binding one agent or antigen to the partial or complete exclusion or other antigens or agents. By binding is meant a detectable binding at least about 1.5 times the background of the assay method. For selective or specific binding such a detectable binding can be detected for a given antigen or agent but not a control antigen or agent. Thus, for example, an agent that selectively binds the IL-20R1/IL-20R2 receptor complex will not bind the IL-22R1/IL-20R2 receptor complex, and vice versa. Further, an agent that selectively binds the IL-20R1/IL-20R2 receptor complex can do so to the partial or complete exclusion of known agents that bind the receptor, e.g., the cytokines IL-20 and/or 11-24. Similarly, an agent that selectively binds IL-22R1/IL-20R2 receptor complex can do so to the partial or complete exclusion of IL-20 and/or IL-24. Furthermore, selectively binds optionally refers to binding a complex but neither component of the complex alone.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

It is understood that the nucleic acids that can encode those peptide, polypeptide, or protein sequences, variants and fragments thereof are also disclosed. This would include all degenerate sequences related to a specific polypeptide sequence, i.e., all nucleic acids having a sequence that encodes one particular polypeptide sequence as well as all nucleic acids, including degenerate nucleic acids, encoding the disclosed variants and derivatives of the polypeptide sequences. Thus, while each particular nucleic acid sequence may not be written out herein, it is understood that each and every sequence is in fact disclosed and described herein through the disclosed polypeptide sequence.

As with all peptides, polypeptides, and proteins, including fragments thereof, it is understood that additional modifications in the amino acid sequence of the provided agents that are polypeptides can occur that do not alter the nature or function of the peptides, polypeptides, or proteins. For example, the provided agents inhibit or reduce the activity of IL-20 and/or IL-24. Modifications that do not alter the function of the agents can occur. Such modifications include, for example, conservative amino acids substitutions and are discussed in greater detail below.

Thus, the provided agents comprising polypeptides or nucleic acids can be further modified and varied so long as the desired function is maintained. It is understood that one way to define any known modifications and derivatives, or those that might arise, of the disclosed nucleic acid sequences and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the polypeptides provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol Biol. 48: 443 (1970), by the search for similarity method of Pearson and Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989); Jaeger et al., Proc. Natl. Acad. Sci. USA 86:7706-7710 (1989); Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism), may arise due to environmental influence (e.g., exposure to ultraviolet light), or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. Amino acid substitutions are typically of single residues but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residue inserted in its place. Such substitutions generally are made in accordance with the following Table 1 and are referred to as conservative substitutions.

TABLE 1
Amino Acid Substitutions

| Amino | Substitutions |
|---|---|
| Acid | (others are known in the art) |
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. By way of example, modifications are made by site specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

The provided compositions can comprise one or more of the agents described herein. By way of example, the compositions can comprise an agent that selectively binds an IL-20R1/IL20-R2 receptor complex, an agent that selectively binds an IL-22R1/IL-20R2 receptor complex, an agent that selectively binds IL-20 and IL-24, and combinations thereof. Thus, the composition can comprise one or more agents that selectively bind an IL-20R1/IL-20R2 receptor complex and one or more agents that selectively bind an IL-22R1/IL-20R2 receptor complex. Optionally, the composition comprises one or more agents that selectively bind an IL-20R1/IL-20R2 receptor complex and one or more agents that selectively bind an IL-20 and IL-24. Optionally, the composition comprises one or more agents that selectively bind an IL-22R1/IL-20R2 receptor complex and one or more agents that selectively bind an IL-20 and IL-24. Thus, the provided compositions can comprise any number of agents and any combination of agents described herein as desired as long as the agents inhibit or reduce the activity of IL-20 and/or IL-24.

The provided compositions are, optionally, suitable for formulation and administration in vitro or in vivo. Optionally, the compositions comprise one or more of the provided agents and a pharmaceutically acceptable carrier. Suitable carriers and their formulations are described in Remington: The Science and Practice of Pharmacy, 21^{st} Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy.

Preparations for parenteral administration include sterile aqueous or non-aqueous solutions, suspensions, and emulsions. Examples of non-aqueous solvents are propylene glycol, polyethylene glycol, oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally used.

Compositions can be formulated to provide quick, sustained or delayed release after administration by employing procedures known in the art. Certain carriers may be more preferable depending upon, for instance, the route of administration and concentration of composition being administered. Suitable formulations for use in the provided compositions can be found in Remington: The Science and Practice of Pharmacy, 21^{st} Edition, David B. Troy, ed., Lippicott Williams & Wilkins (2005).

Provided herein are kits comprising one or more of the provided compositions and instructions for use. Optionally, the kit comprises one or more doses of an effective amount of a composition comprising an agent that inhibits or reduces the activity of IL-20 and/or IL-24. Optionally, the composition is present in a container (e.g., vial or packet). Optionally, the kit comprises one or more additional agents for treating or preventing one or more symptom of an inflammatory and/or autoimmune disease. Optionally, the kit comprises a means of administering the composition, such as, for example, a syringe, needle, tubing, catheter, patch, and the like. The kit may also comprise formulations and/or materials requiring sterilization and/or dilution prior to use.

The provided compositions comprising one or more agents that inhibit or reduce the activity of IL-20 and/or IL-24 can be used to treat or prevent one or more symptom of an inflammatory and/or autoimmune disease. Thus, provided herein are methods of treating or preventing an inflammatory or autoimmune disease in a subject. The methods include administering an effective amount of the compositions, wherein administering the effective amount of the composition treats or prevents the inflammatory or autoimmune disease in the subject. Optionally, the disease is psoriasis or rheumatoid arthritis.

Optionally, the provided methods include administration of one or more additional agents that treat or prevent the inflammatory or autoimmune disease. For example, the provided methods can further include administration of and effective amount of one or more of anti-inflammatory agents. Suitable additional agents for use in the provided methods include, but are not limited to, analgesics, non-steroidal anti-inflammatory drugs, disease-modifying anti-rheumatic drugs, corticosteroids, and vitamin D analogues. Exemplary disease-modifying anti-rheumatic drugs for treating or preventing rheumatoid arthritis include, but are not limited to, azathioprine, ciclosporin (cyclosporine A), D-penicillamine, gold salts, hydroxychloroquine, leflunomide, methotrexate (MTX), minocycline, sulfasalazine (SSZ), and cyclophosphamide. Other agents for use in treating or preventing psoriasis include, but are not limited to, anthralin, retinoids, calcineurin inhibitors, salicylic acid and coal tar.

Combinations of agents or compositions can be administered either concomitantly (e.g., as a mixture), separately but simultaneously (e.g., via separate intravenous lines or other administration means) or sequentially (e.g., one agent is administered first followed by administration of the second agent). Thus, the term combination is used to refer to concomitant, simultaneous or sequential administration of two or more agents or compositions.

The methods and agents as described herein are useful for both prophylactic and therapeutic treatment. For prophylactic use, a therapeutically effective amount of the agents described herein are administered to a subject prior to onset (e.g., before obvious signs of inflammatory or autoimmune disease) or during early onset (e.g., upon initial signs and symptoms of inflammatory or autoimmune disease). Prophylactic administration can occur for several days to years prior to the manifestation of symptoms of disease. Prophylactic administration can be used, for example, in the preventative treatment of subjects diagnosed with a genetic predisposition to disease. Therapeutic treatment involves administering to a subject a therapeutically effective amount of the agents described herein after diagnosis or development of disease.

According to the methods taught herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., autoimmune disease, viral infection, or cancer). The term patient or subject includes human and veterinary subjects.

Provided herein are methods of screening for agents that selectively bind the IL-20R1/IL-20R2 receptor complex. The methods include contacting an agent to be screened with the IL-20R1/IL-20R2 receptor and determining whether the agent binds the IL-20R1/IL-20R2 complex. Optionally, the agent contacts one or more of the amino acids selected from the group consisting of Gly-224, Arg-128, Gly-77, Tyr-76, Glu-105, Gln-107, Pro-225 and Pro-226 of IL-20R1. Optionally, the agent contacts one or more of the amino acids selected from the group consisting of Glu-164, Arg-133, Tyr-74, Glu-75 and Thr-104 of IL-20R2. Also provided are methods of screening for agents that selectively bind IL-22R1/IL-20R2 receptor complex, which methods include contacting an agent to be screened with the IL-22R1/IL-20R2 receptor and determining whether the agent binds the IL-22R1/IL-20R2 complex. It is understood and contemplated herein that numerous methods may be used to detect the binding of an agent to a receptor. For example, binding can be detected directly by assaying coupling between an agent and a receptor. Binding can be determined, for example, by selecting an assay from the group consisting of a coimmunoprecipitation assay, a colocalization assay, or a fluorescence polarizing assay, as described below. The assays are known in the art, e.g., see Sambrook et al., Molecular Cloning: A Laboratory Manual, 3^{rd} Ed., Cold Spring Harbor Press, Cold Spring Harbor, N.Y. (2001); Dickson, Methods Mol. Biol. 461:735-44 (2008); Nickels, Methods 47(1):53-62 (2009); and Zinchuk et al., Acta Histochem. Cytochem. 40(4):101-11 (2007). By way of another example, binding can be detected by determining whether the agent to be tested competitively inhibits a molecule known to bind to the receptor. Thus, in the provided screening methods, the determining step optionally comprises determining whether the agent prevents or reduces binding of IL-20 and/or IL-24 to the IL-20R1/IL-20R2 complex.

Alternatively, or additionally, the detection of binding can be determined by assaying the presence of down-stream molecules or events. Thus, optionally, binding indicates the agent inhibits or reduces the activity of IL-20. Optionally, binding indicates the agent further inhibits or reduces the activity of IL-24. For example, the ability of the agent to be tested to selectively bind the IL-20R1/IL-20R2 receptor can be determined by assaying for the ability of the agent to inhibit or reduce the activity of IL-20 using a 3-D human epidermis model as described in Wolk et al., J. Mol. Med. (Berl.) 87(5):523-36 (2009), which is incorporated by reference herein in its entirety. By way of another example, the ability of the agent to be tested to selectively bind the IL-20R1/IL-2082 receptor can be determined by assaying for the ability of the agent to inhibit or reduce the activity of IL-20 using a mouse model that overexpresses IL-20 as described in Blumberg et al., Cell 104:9-19 (2001). Thus, in the provided screening methods, the contacting step optionally comprises administering an agent to be a tested to a subject (e.g., a mouse) and the determining step comprises determining whether the agent inhibits or reduces the activity of IL-20 and/or IL-24, wherein inhibition or reduction of activity indicates the agent selectively binds the IL-20R1/IL-20R2 or the IL-22R1/IL-20R2 receptor complex.

Binding can be assessed by determining if the agent reduces the severity of one or more symptoms of the autoimmune or inflammatory disease or condition. Thus, by way of example, in the provided screening methods, the contacting step comprises administering the agent to a subject with an autoimmune or inflammatory disease and the determining step comprises determining whether the agent prevents or reduces one or more symptoms of the disease in a subject. Optionally, the disease is psoriasis or rheumatoid arthritis. Such screening methods can be carried out using, for example, animal models of inflammatory and autoimmune disease, which can be obtained from Jackson Laboratory, 600 Main Street, Bar Harbor, Me. 04609 USA.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a composition is disclosed and discussed and a number of modifications that can be made to a number of molecules including the composition are discussed, each and every combination and permutation of the composition, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims below.

### EXAMPLES

### Example 1

### Targeted Modulators of IL-20 Family Cytokines

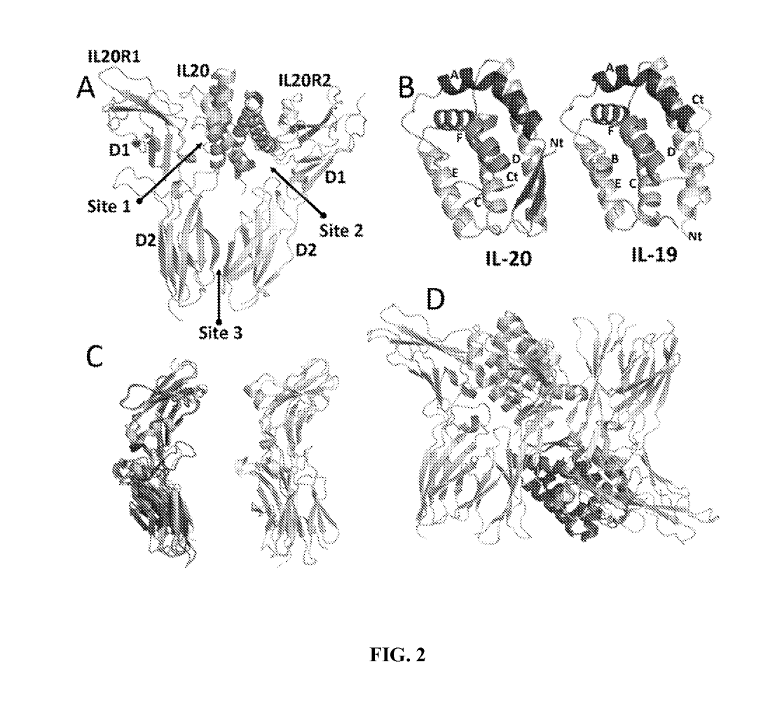

The crystal structure of IL-20 bound to the extracellular domains of the IL-20R1 and IL-20R2 chains (IL-20/IL-20R1/IL-20R2) is shown in FIG. 2. This is the first ternary complex structure for this family of cytokines, which play important roles in protecting the host from pathogens. Dysregulation of these cytokines is implicated in pathology; specifically psoriasis and rheumatoid arthritis. The structure of IL-20/IL-20R1/IL-20R2 provided herein shows the structural-basis for receptor sharing and specificity between the IL-10 family members (FIG. 1). Based on this structure, receptor heterodimers that specifically neutralize IL-20 and IL-24 biological activity were designed. Such molecules provide antagonists for treating psoriasis and/or rheumatoid arthritis, or other inflammatory diseases. Formation of a soluble IL-22R1/IL-20R2 receptor heterodimer yielded a soluble antagonist specific for IL-20 and IL-24. See FIG. 4.

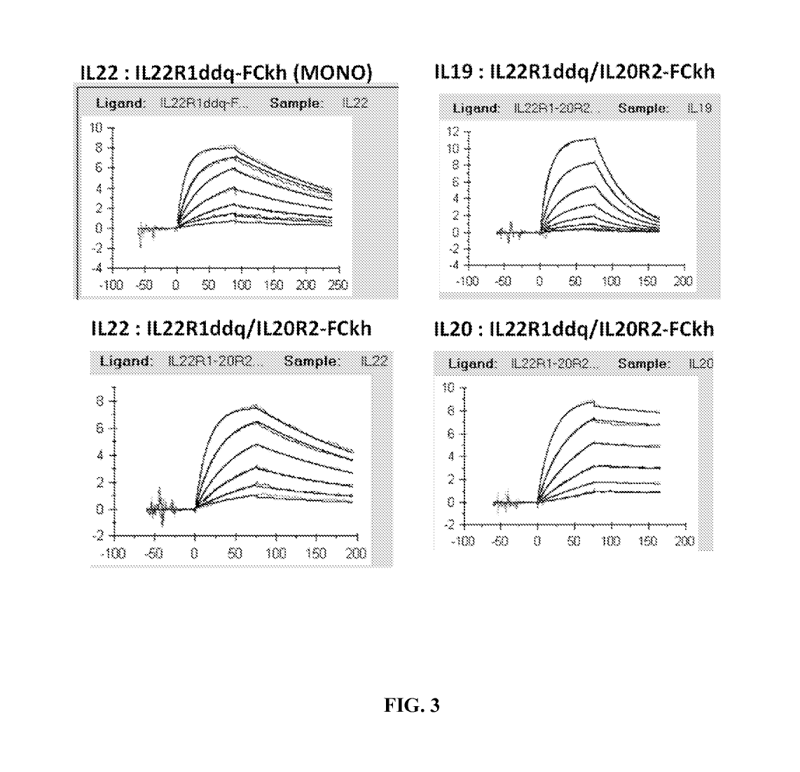

FIG. 3 shows the analysis of IL-19, IL20, IL-22 binding to IL-22R1/IL-20R2-FCkh and IL-22 binding to IL-22R1. Binding studies with the IL-22R1/IL-20R2-FCkh heterodimer revealed this complex also binds IL-22 with high affinity (FIGS. 3A and 3B). Surface Plasmon resonance (SPR) sensorgram data are shown in FIGS. 3A-3D. Kinetic parameters are shown in Table 2 below. The IL-22R1 used herein contains asparagines Asn-80^{IL-22R1} and Asn-87^{IL-22R1} replaced by aspartic acids and Thr-89^{sIL-22R1} replaced by glutamine to make sIL-22R1_{DDQ} (Jones et al., Structure 16(9):1333-44 (2008)).

TABLE 2
Kinetic Parameters of Analyte Ligand Binding.

| Analyte | Ligand | Kon | Koff | KD |
|---|---|---|---|---|
| IL22 | IL22R1ddq-FCkh (MONO) | 1.47E+06 | 0.004918 | 3.34 nM |
| IL22 | IL22R1ddq/IL20R2-FCkh | 0.75E+05 | 0.004926 | 5.15 nM |
| IL20 | IL22R1ddq/IL20R2-FCkh | 7.84E+05 | 7.76E−04 | 0.99 nM |
| IL19 | IL22R1ddq/IL20R2-FCkh | 2.17E+05 | 0.0218 | 100.6 nM |

Table 2 and FIG. 3 show IL-19 and IL-20 exhibit a 100-fold difference in binding affinity for IL-22R1/IL-20R2. However, IL-22 and IL-20 bind to the heterodimer with almost identical strength. A description of the ligands is shown in FIGS. 4A-4D.

Based on this information, soluble IL-20R1/IL22R1 hybrid receptors plus IL20R2 receptor-FC heterodimers were designed to selectively neutralize IL-20 and IL-24. Two such exemplary hybrid receptors are shown in FIGS. 4C and 4D. The IL-20R1/IL-22R1 and IL-22R1/IL-20R1 hybrids were paired with IL-20R2 to form receptor heterodimers with selective specificity for IL-20 and IL-24 (FIGS. 4C and 4D). These hybrid receptors contain essentially the D1 domain of IL-20R1, or IL-22R1, with the D2 domain of IL-22R1, or IL-20R1 (FIGS. 1 and 4C and 4D). The hybrid receptors required the crystal structure of the IL-20/IL-20R1/IL-20R2 and IL-22/IL-22R1 to design. Additional mutations in site 3 of the receptors can further optimize the specificity and affinity of the receptor hybrids.

In addition to receptor hybrids, IL-20 mutants were created that specifically target the IL-20R1/IL-20R2 receptor complex. These molecules can confirm the mechanism of IL-19/IL-20 specificity. Further, an IL-20 specific for IL-20R1/IL-20R2 can be used to understand the biology of IL-20 signaling through the IL-20R1/IL-20R2 complex (e.g. no IL-22R1/IL-20R2 signaling) and/or treat disease by also specifically antagonizing IL-20R1/IL-20R2 signaling.

Creation of an IL-19/IL-20R1/IL-20R2 structural model, based on the crystal structure of IL-19 and IL-20/IL-20R1/IL-20R2 crystal structures, reveals IL-19's unique specificity is determined by the C-terminus of IL-19. The structure comparison of IL-19 and IL-20 revealed the C-terminus of IL-19 is structurally identical to the N-terminus of IL-20. Based on this analysis, IL-20 mutants that selectively bind to the IL-20R1/IL-20R2 complex were designed and are shown below.

The sequence alignment between human IL-19 (amino acids 6-161 of SEQ ID NO:1) and human IL-20 (amino acids 1-153 of SEQ ID NO:3) is as follows.

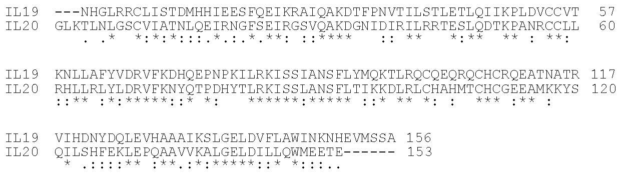

N-terminal sequences of wildtype (WT) IL-20 is (GLKTLNLGSC (SEQ ID NO:35)). If the IL-20 is expressed in E. coli, the “G” is replaced by an “M.” Amino acid changes at the N-terminus of IL-20 make it specific for the IL-20R1/IL-20R2 complex, excluding it from binding the IL-22R1/IL-20R2 complex. The sequence for the first mutant (M1) is ASSMKTLNLGSC (SEQ ID NO:36). The sequence for the second mutant M2 is CSSMKTLNLGSC (SEQ ID NO:37). The mutant M2 also comprises an additional mutation, in the glutamine (Q), bolded above, at position 17 of SEQ ID NO:3. This N-terminal segment disrupted IL-22R1 binding.

The full sequences of IL-20M1 and IL-20M2 are as follows.

IL-20 M1 sequence
(SEQ ID NO: 38)
ASSMKTLNLGSCVIATNLQEIRNGFSEIRGSVQAKDGNIDIRILRRTESL
QDTKPANRCCLLRHLLRLYLDRVFKNYQTPDHYTLRKISSLANSFLTIKK
DLRLCHAHMTCHCGEEAMKKYSQILSHFEKLEPQAAVVKALGELDILLQW
MEETE
IL-20 M2 sequence
(SEQ ID NO: 39)
CSSMKTLNLGSCVIATNLCEIRNGFSEIRGSVQAKDGNIDIRILRRTESL
QDTKPANRCCLLRHLLRLYLDRVFKNYQTPDHYTLRKISSLANSFLTIKK
DLRLCHAHMTCHCGEEAMKKYSQILSHFEKLEPQAAVVKALGELDILLQW
MEETE

The sequence of an IL-19/IL-20 hybrid molecule to selectively bind the IL-20R1/IL-20R2 complex can comprise or consist of the following sequence (wherein the bolded sequence is IL-19 and the remainder is IL-20):

IL19/IL20 hybrid
(SEQ ID NO: 21)
MNHGLRRCLISTDLQEIRNGFSEIRGSVQAKDGNIDIRILRRTESLQDTK
PANRCCLLRHLLRLYLDRVFKNYQTPDHYTLRKISSLANSFLTIKKDLRL
CHAHMTCHCGEEAMKKYSQILSHFEKLEPQAAVVKALGELDILLQWMEEH
EVMFSA

An IL-19/IL-20 hybrid can also comprise or consist of the following sequence (which includes an additional histidine residue at the C-terminal):

(SEQ ID NO: 45)
HMNHGLRRCLISTDLQEIRNGFSEIRGSVQAKDGNIDIRILRRTESLQDT
KPANRCCLLRHLLRLYLDRVFKNYQTPDHYTLRKISSLANSFLTIKKDLR
LCHAHMTCHCGEEAMKKYSQILSHFEKLEPQAAVVKALGELDILLQWMEE
HEVMFSA.

It should be noted that any of the sequences shown throughout the application can similarly include or exclude one or more histidine residues at the C-terminal and can include or exclude one or more histidine residues at the N terminal. Such histidine residues can be enzymatically cleaved using methods routine in the art.

In addition to molecules that specifically bind the IL-20R1/IL-20R2 complex, IL-22 mutant molecules were created that specifically bind to the IL-22R1/IL-20R2 receptor complex. The structure of the IL-20/IL-20R1/IL-20R2 complex provided herein and structure of IL-22 allow the design of an IL-22 molecule that selectively binds to the IL-22R1/IL-20R2 complex (e.g. no IL-20R1/IL-20R2 signaling). This designed cytokine can specifically target the IL-22R1/IL-20R2 complex; either activating signaling through the complex or preventing signaling through the complex. This molecule is unique because it targets the heterodimeric complex (IL-22R1/IL-20R2) and not a single receptor chain. The structure of IL-20/IL-20R1/IL-20R2 provided herein allows a way to design an IL-22 that can bind IL-20R2. Part of the wildtype IL-22 sequence is as follows.

(SEQ ID NO: 40)
HCRLDKSNFQQPYITNRTFMLAKEASLADQNTDVRLIGEKLFHGVSMSER
CYLMKQVLQFTLEEVLFPQSDRFQPYMQEVVPFLARLSNRLSTCHIEGDD
LHIQRNVQKLKDTVKKLGESGEIKAIGELDLLFMSLRNACI

Residues underlined correspond to the CD loop region of IL-22 that prevents the molecule from binding to IL-20R2. In particular, the phenylalanine (F) (in bold font at position 73 of SEQ ID NO:40) is important in this specificity of IL-22.

One hybrid IL-22 sequence designed to prevent IL-10R2 binding comprises a Y to A mutation (in bold font, italics and lower case) and replacement of the IL-22 amino acid sequence underlined above with the amino acid sequence underlined in the sequence shown below. Lower case letters correspond to new residues, derived from IL-20. Capital letters in the underlined region are from the IL-22 sequence.

(SEQ ID NO: 41)
HCRLDKSNFQQP [symbol] ITNRTFMLAKEASLADQNTDVRLIGEKLFHGVSMSER
CYLMKQVLQFTLEEVfknyqtpshdYMssVanFLARLSNRLSTCHIEGDD
LHIQRNVQKLKDTVKKLGESGEIKAIGELDLLFMSLRNACI

### Example 2

### Structural Basis for Receptor Sharing and Activation by IL-20R2 Binding Cytokines

IL-20 has emerged as a highly pleotropic cytokine involved in essential cellular processes and pathology. Despite advances an improved understanding of IL-20 biology, the molecular mechanisms that allow IL-20, IL-19, and IL-24 to discriminate and activate the type I (IL-20R1/IL-20R2) and type II (IL-22R1/IL-20R2) receptor complexes are unknown. To address this question the crystal structure of the IL-20/IL-20R1/IL-20R2 complex was determined to evaluate how signaling complexity can be obtained for structurally similar cytokines

Results

Overall Architecture of the IL-20/IL-20R1/IL-20R2 Signaling Complex

The structure of IL-20/IL-20R1/IL-20R2 (FIG. 2A) was solved at 2.8 [symbol] by SAD phasing and molecular replacement methods. The IL-20/IL-20R1/IL-20R2 data collection and refinement statistics are as follows: data collection statistics—space group P2₁2₁2₁; a (Å) 103.12; b ( [symbol] ) 111.76; c ( [symbol] ) 136.33; resolution ( [symbol] ) 50-2.8; number of reflections 38,635; completeness (%) 98.9 (90.7); redundancy 8.4 (6.4); I/sigma 41.7 (4.8); R_{sym}(%) 7.0 (34.2); values in parentheses correspond to the highest resolution shell (2.9 [symbol] -2.8 [symbol] ); refinement statistics: resolution ( [symbol] ) 50-2.8; R_{work} (%) 20.9; No. reflections (work) 36,043; R_{free} (%) 27.4; No. reflections (free) 1,962; Residues in model IL-20: 24-176, Chains A, C, IL-20R2: 34-215, Chains B, D, IL-20R1: 24-227, Chains R, E; No. protein atoms 8,826; No. water atoms 57; No. carbohydrate atoms 37; RMSD bond distance (Å) 0.009; RMSD bond angles (°) 1.43; Average B factor (Å²) 37.3; Ramachandran plot, Most favored (%) 85.2; Additionally allowed (%) 14.0; Generally allowed (%) 0.4; Disallowed (%) 0.4.

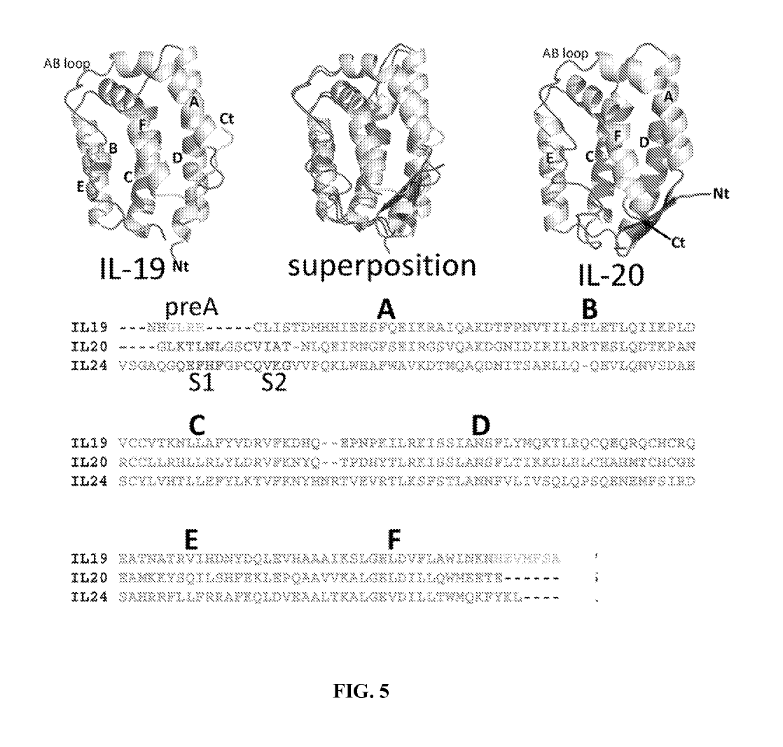

IL-20 adopted an α-helical fold, which was highly conserved with IL-19 (r.m.s.d. 0.79 [symbol] ). Despite structurally similar helical cores, the N-terminus of IL-20 adopted a novel β-hairpin structure, rather than the 3¹⁰ helix/coil structure observed in IL-19 (FIG. 2B). Threading the IL-24 amino acid sequence onto the structure of IL-20 suggested IL-20 and IL-24 adopt similar N-terminal β-hairpin structures, which positions Cys-59^{IL-24} and Cys-106^{IL-24} in close proximity (Cα-Cα distance=6.6 [symbol] ) for disulfide bond formation (FIG. 5). Thus, the predicted structural similarity of IL-20 and IL-24 was consistent with the identical receptor binding profiles of each cytokine (FIG. 1).

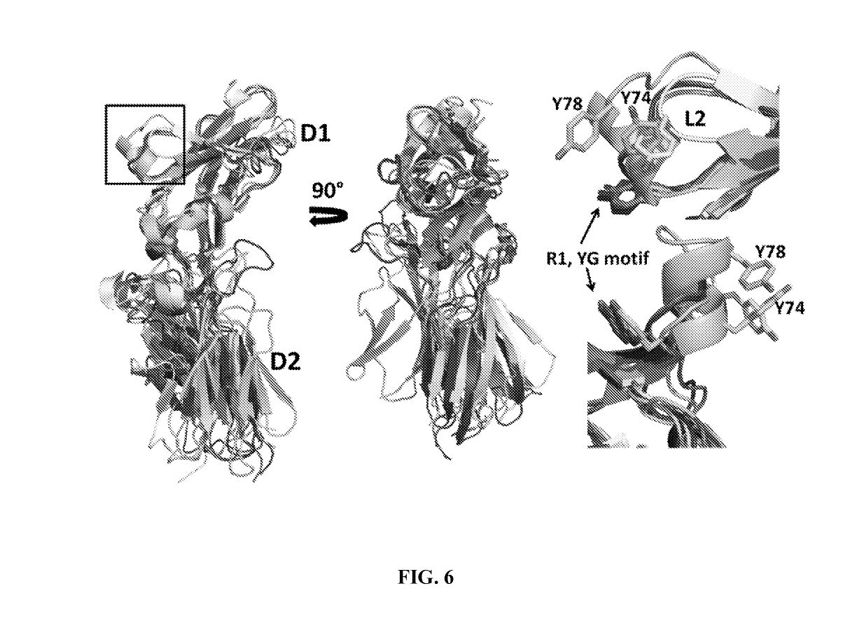

The extracellular fragments of IL-20R1 and IL-20R2 each consist of tandem β-sandwich domains (D1 and D2) that assemble around IL-20 to form a V-shaped complex, when viewed down the α-helical bundle axis of IL-20 (FIG. 2A). X-ray analysis of IL-20R1 and IL-20R2 completed the structural descriptions of all five IL-10 family receptors (23-26) (FIG. 1). Structural comparisons of the receptors revealed the three R1 chains were highly similar to one another, while IL-10R2 and IL-20R2 exhibit structurally divergent cytokine binding loops and inter-domain angles (FIGS. 2C and 6).

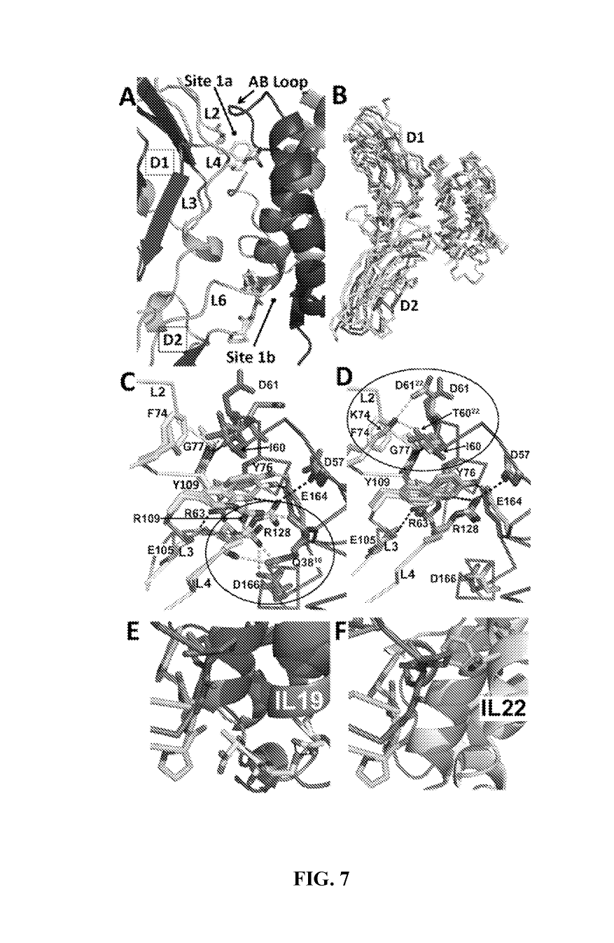

IL-20/IL-20R1/IL-20R2 ternary complex formation was mediated by 3 protein interfaces, IL-20/IL-20R1 (site 1), IL-20/IL-20R2 (site 2), and IL-20R1/IL-20R2 (site 3), that bury a total of 4,236 [symbol] ² of accessible surface area (FIG. 2A). The IL-20/IL-20R1 site 1 interface (1,576 [symbol] ²) consisted of two contact surfaces, site 1a and site 1b (FIG. 7A). Site 1a was formed by IL-20R1 L2-L4 loops that contact a small cavity on IL-20 located at the intersection of helix F and AB loop. Site 1b contacts occurred between IL-20R1 L6 and the N-terminus of helix A. Site 1a contributed ˜83% of the total buried surface area and 8 of 9 hydrogen bond/salt bridge interactions identified in the IL-20/IL-20R1 interface (Table 3, FIG. 7).

TABLE 3
Hydrogen Bonds in the IL-20/IL-20R1/IL-20R2 Complex
IL-20/IL-20R1, Site 1 Interface

| IL-20 | | | | IL-20R1 | | | Contact Conserved? | |
|---|---|---|---|---|---|---|---|---|
| Res# | Res | Atom | Atom | Res | Res# | Dist(Å) | | |
| 40 | GLN | NE2 | O | GLY | 224 | 3.1 | | |
| 57 | ASP | OD2 | NH2 | ARG | 128 | 2.7 | | |
| 60 | ILE | O | N | GLY | 77 | 3.1 | IL-10, IL-22, IL-19 | |
| 62 | ILE | O | OH | TYR | 76 | 3.3 | IL-10, IL-22, IL-19 | |
| 63 | ARG | NH1 | O | GLU | 105 | 3.3 | IL-22 | |
| 63 | ARG | NH1 | OE1 | GLN | 107 | 3.5 | | |
| 67 | ARG | NE | OE1 | GLU | 105 | 3.4 | | |
| 160 | LYS | NZ | OH | TYR | 76 | 2.9 | IL-10, IL-22, IL-19 | |
| 164 | GLU | OE2 | OH | TYR | 76 | 2.5 | IL-10, IL-22, IL-19 | |
| IL-20/IL-20R2 Site 2 Interface | | | | | | | Additional Contacts in IL- | |
| IL-20 | | | IL-20R2 | | | | 19/IL-20R2 Interface | |
| Res# | Res | Atom | Atom | Res | Res# | Dist(Å) | IL-19 | IL-20R2 |
| 24 | GLY | N | OE1 | GLU | 164 | 2.9 | OD1 ASP 38 . . . LYS 210 NZ | |
| 41 | GLU | OE2 | NH2 | ARG | 133 | 2.9 | NE2 HIS 41 . . . THR 104 O** | |
| 41 | GLU | OE2 | NH1 | ARG | 133 | 3.4 | OD1 ASP 92 . . . YR 78 OH | |
| 41 | GLU | OE1 | NH2 | ARG | 133 | 3.1 | OD1 GLN 120 TYR 78 OH | |
| 91 | LEU | O | OH | TYR | 74 | 2.9 | O SER 158 . . . ARG 184 NH1 | |
| 96 | LYS | N | OH | TYR | 74 | 3.1 | | |
| 110 | SER | OG | OE1 | GLU | 75 | 3.3 | | |
| 110 | SER | OG | OE2 | GLU | 75 | 2.6 | | |
| 111 | SER | OG | OG1 | THR | 104 | 2.6 | | |
| 114 | ASN | OD1 | N | TYR | 74 | 2.9 | | |
| 114 | ASN | ND2 | OE1 | GLU | 75 | 3.0 | | |
| 114 | ASN | ND2 | OE2 | GLU | 75 | 3.5 | | |
| 121 | LYS | NZ | OE1 | GLU | 73 | 2.7 | | |

IL-20R1/IL-20R2 Site 3 Interface

| IL-20R1 | | | IL-20R2 | | | |
|---|---|---|---|---|---|---|
| Res# | Res | Atom | Atom | Res | Res# | Dist(Å) |
| 187 | ASN | ND2 | OD1 | ASP | 148 | 3.5 |
| 190 | SER | OG | OD2 | ASP | 148 | 3.1 |
| 195 | SER | N | O | PRO | 189 | 3.0 |
| 195 | SER | O | N | HIS | 191 | 3.2 |
| 206 | THR | O | NE2 | HIS | 151 | 2.7 |
| 207 | TRP | O | NE2 | HIS | 151 | 3.4 |

IL20/IL20R2 Dimer Interface

| | | |
|---|---|---|
| IL20 | IL20R2 | IL20R2 |

| Res# | Res | Atom | Atom | Res | Res# | Dist(Å) | Atom | Res | Res# | Dist(Å) |
|---|---|---|---|---|---|---|---|---|---|---|
| 120 | LYS | O | OH | TYR | 215 | 3.5 | | | | |
| 124 | ARG | NH2 | OE1 | GLU | 178 | 2.8 | | | | |
| | | | *OE1 | GLN | 134 | . . . | NE2 | GLN | 134 | 3.0 |

Underlined contacts in the IL-20/IL-20R2 site 2 interface were conserved in IL-19 and IL-24 site 2 interfaces.
**This IL-19/IL-20R2 contact replaced IL-20/IL-20R2 contacts 2-4 in the table.

In the IL-20/IL-20R2 dimer interface, *Gln-134 is an Asn, which formed an N-linked glycosylation site in IL-20R2WT protein.

In contrast to site 1a, site 1b was very small and made up almost entirely of IL-20R1 L6 residues Gly-224^{IL-20R1}, Pro-225^{IL-20R1}, and Pro-226^{IL-20R1}, which formed Van der Waal contacts with the aliphatic portion of Arg-43^{IL-20} and a single hydrogen with helix A residue Gln-40^{IL-20}.

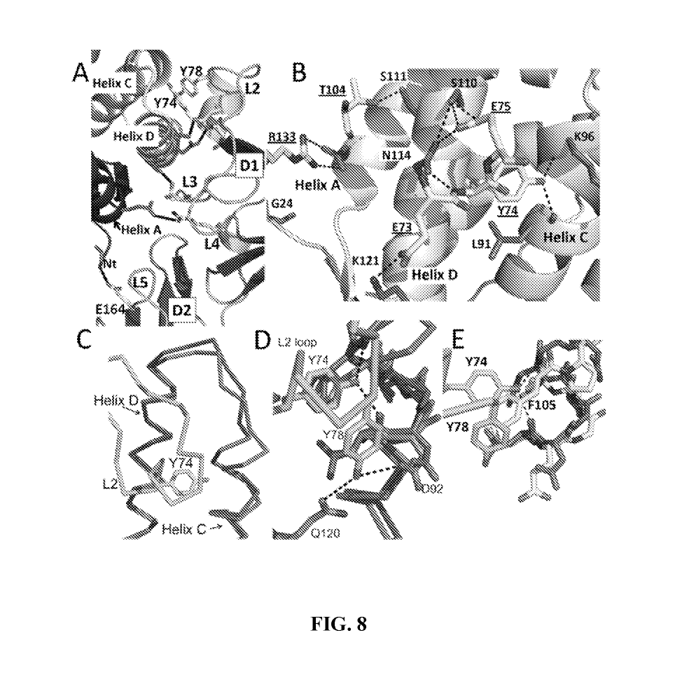

The site 2 IL-20/IL-20R2 interface (1,624 [symbol] ²) was centered on IL-20 helix D, which is surrounded by IL-20R2 L2 and L3 loops (FIG. 8). The IL-20R2 L2 loop also made significant contacts with helix C, via Tyr-74^{IL-20R2}, while residues on L4, and L5 loops formed hydrogen bonds with helix A and the IL-20 N-terminus. As observed in site 1a, the IL-20R2 L2 loop formed the majority of contacts in site 2 by contributing 57% of the buried surface area, and 9 of 13 hydrogen bonds in the IL-20/IL-20R2 interface (Table 3). Six of these hydrogen bonds were formed with helix D residues Ser-110^{IL-20}, Ser-111^{IL-20}, and Asn-114^{IL-20} that are conserved in the sequences of IL-19 and IL-24 (FIGS. 5 and 8B). Thus, IL-20R2, and the IL-20SFCs share a conserved binding epitope to facilitate promiscuous IL-20R2 ligand engagement (FIG. 1).

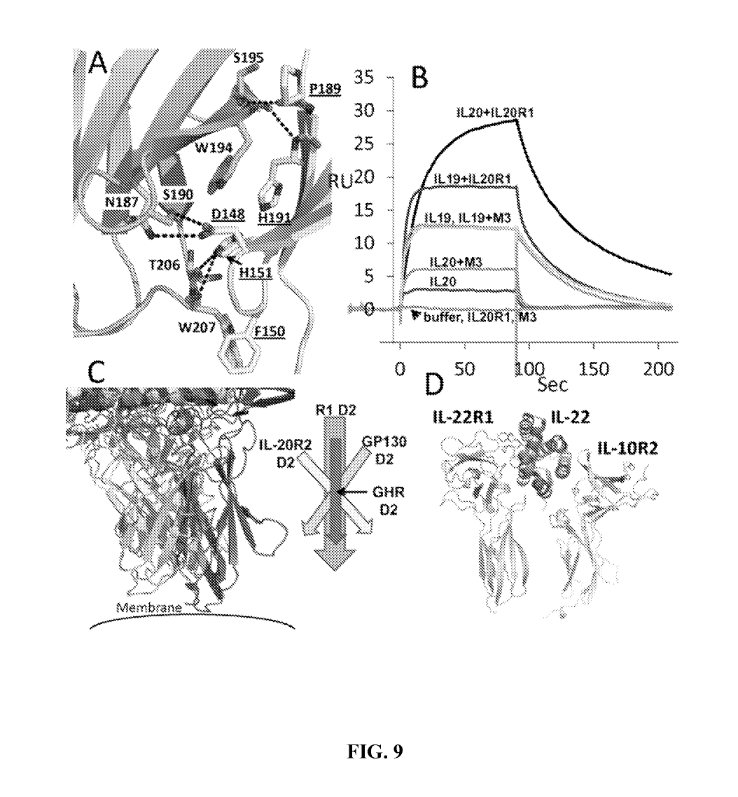

Essentially all contacts between IL-20 and the receptors were mediated by the D1 domains, while IL-20R1 and IL-20R2 D2 domains contact one another to form the base of the V-shaped complex (site 3, FIGS. 2A, 9A). The site 3 interface (1,036 [symbol] ²) was formed from IL-20R1 D2 residues on β-strand C′, the CC′ loop, and the EF loop, which contact IL-20R2 residues on the AB loop, β-strand E, and the EF loop. The interface was quite extensive including 6 hydrogen bonds (Table 3) contributed predominantly from AB loop of IL-20R2 and the EF loop of IL-20R1.

The IL20/IL-20R1/IL-20R2 Dimer Complex

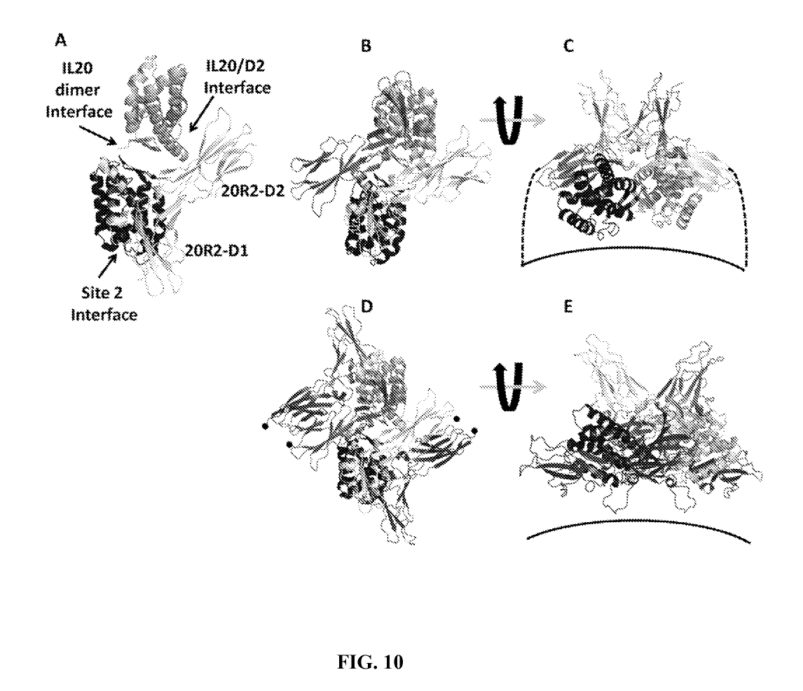

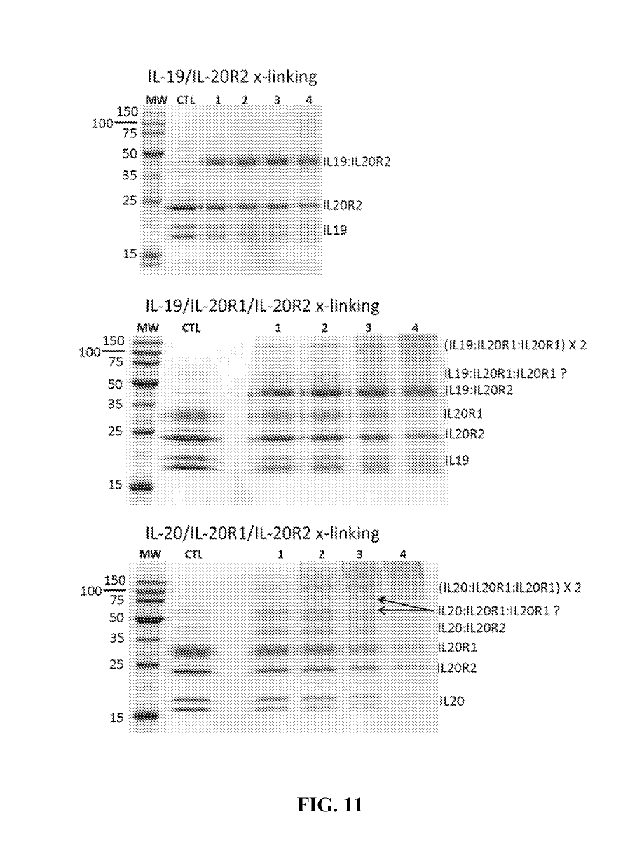

Two 1:1:1 IL-20/IL-20R1/IL-20R2 complexes formed a dimer in the asymmetric unit of the crystals (FIG. 2D). The IL-20 dimer interface was very small (˜200 [symbol] ²), consistent with IL-20 being a monomer in solution. The dimer was stabilized by contacts formed between IL-20R2 chains that bridge the two-fold related IL-20s. Each IL-20R2 formed a site 2 interface (FIGS. 2A and 8), and a second contact between IL-20R2 D2 domain and IL-20 helix D (residues 121-130, FIG. 10). The IL-20/IL-20R2 dimer interface was extensive, burying 1,982 [symbol] ² of accessible surface area. Two IL-20R1 chains were also in the complex, but they do not contribute residues to the dimer interface. Although the formation of cell surface IL-20R2 dimers might provide an explanation for IL-20SFC signaling on immune cells that apparently lack IL-20R1 (Nagalakshmi et al., In. Immunopharmacol. 4:577-592 (2004)), an IL-19/IL-20R2 dimeric complex was not detected in solution, or significant differences in IL-20 SFC binding affinity between monomeric and dimeric IL-20R2 was not identified (FIG. 11, Table 4).

TABLE 4
SPR binding constants for IL-19 and IL-20 Complexes

| Analyte | Ligand | kon (1/sec M) | koff (1/sec) | KD (nM) |
|---|---|---|---|---|
| IL-19 | IL-20R2* | 1.10E+06 | 0.0398 | 36.3 |
| IL-20 | IL-20R2 | | | 869.8 |
| IL-19c** | IL-20R2-fc | 2.30E+05 | 0.0242 | 105.2 |
| IL-20d | IL-20R2-fc | 3.19E+05 | 0 2192 | 686.5 |
| IL-19a | IL-20R1-fc | — | — | — |
| IL-20b | IL-20R-fc | — | — | ~9,000 |
| IL-19 + IL20R1e | IL-20R2-fc | 4.45E+04 | 0.139 | 3,121.0 |
| IL-20 + IL20R1f | IL-20R2-fc | 5.37E+04 | 0.01714 | 319.3 |
| IL-19 + IL22R1g | IL-20R2 | — | — | — |
| IL-20 + IL22R1h | IL-20R2 | 6.55E+04 | 0.08927 | 1,363.0 |

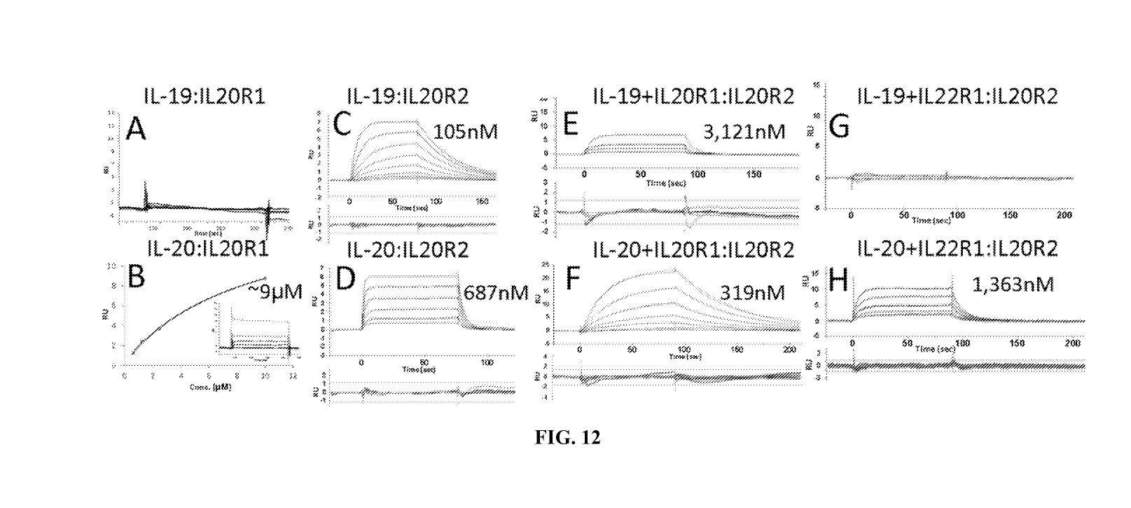

*“IL-20R2” in the ligand column corresponds to amine coupled IL-20R2.
**The letter following the analyte descriptor, corresponds to the sensorgrams shown in FIG. 12.

Mechanisms Regulating IL-20R1 Chain Affinity

Surface plasmon resonance (SPR) experiments were performed to quantify IL-20R1 binding to IL-19 and IL-20 (FIG. 12). Binding was not detected between IL-19 and IL-20R1 at IL-19 concentrations up to 10 μM. However, a binding constant of ˜9 μM was determined for the IL-20/IL-20R1 interaction (FIG. 12B). In contrast to very weak IL-20R1 interactions, IL-10R1 and IL-22R1 chains exhibit ˜10,000-fold tighter binding affinity for IL-10 (kd=0.5 nM) and IL-22 (kd=1.2 nM) (FIG. 1 and Jones et al., Structure 16:1333-44 (2008); Yoon et al., J. Biol. Chem. 281:35088-35096 (2006)). These extremely different binding affinities occur despite structurally similar IL-20/IL-20R1, IL-22/IL-22R1 and IL-10/IL-10R1 binary complexes (FIG. 7B). Comparison of each interface revealed identical “YG” interaction motifs consisting of receptor YG residues (L2 loop Tyr-76^{sIL-20R1} and Gly-77^{sIL-20R1} in IL-20R1, FIG. 7A) that are inserted into a conserved cleft formed by the AB loop and helix F of the ligands (FIGS. 7C, 7D, refs. Josephson et al., Immunity 15:35-46 (2001); Jones et al., Structure 16:1333-44 (2008); Bleicher et al., FEBS Lett. 582:2985-2992 (2008). Buried surface area did not predict the observed affinities of the different complexes (Table 5).

TABLE 5
Comparison of IL-10 Family Site 1 and Site 2 Interface Parameters

| | 10R1: IL-10 | 22R1: IL-22 | 20R1: IL-20 | 20R2: IL-20 |
|---|---|---|---|---|
| | SITE 1 | SITE 1 | SITE 1 | SITE 1 |
| SITE KD | 0.57 nM^{a} | 1.2 nM^{b} | 91 μM | 687 nM |
| buried surface* | 909 Å | 801 Å | 794 Å | 830 Å |
| No. Residues* | 27 | 25 | 21 | 21 |
| No. H-bonds | 15 | 12 | 9 | 13 |

a, from reference (He and Liang, J. Immunol. 184:1793-8 (2010); b, from reference Hsu, J. Exp. Med. 208:1849-1861 (2010)

*buried surface, and number of residues, corresponds to the ligands only.

However, complex affinities correlated with unique contacts made by four receptor residues (D1 residues Phe-74 (L2 loop), Glu-105 (L3 loop), Tyr-109, and Arg-128 (L4 loop) in IL-20R1) located adjacent to the YG interaction motif (FIGS. 7C and 7D).

In the high affinity IL-10/IL-10R1 complex, Arg-109^{IL-10R1} and Arg-128^{IL-10R1}, adjacent to the YG motif, formed a network of four hydrogen bonds with IL-10 residues Gln-38^{IL-10} and Asp-166^{IL-10} (FIG. 7C). However, in IL-20/20R1 and IL-22/IL-22R1 interfaces, Arg109^{IL-10R1} was replaced with Tyr-109^{IL-20R1, IL-22R1}, which no longer contacted IL-20 or IL-22 directly, but re-positioned Arg-128^{IL-20R1, IL-22R1}, for interactions with Asp-57^{IL-20, IL-22}, despite the fact Asp-166 was conserved in IL-20 and IL-22 (FIG. 7D). Thus, this network of four hydrogen bonds in the IL-10/IL-10R1 interface was reduced to one in IL-20/IL-20R1 and IL-22/IL-22R1 interfaces.

To compensate for the loss of an “IL-10 like” bonding network, the IL-22/IL-22R1 interface formed a unique 3 hydrogen bond network on the opposite side of the YG motif (FIG. 7D). This interaction included hydrogen bonds between Arg-63^{IL-22} and Glu-105^{IL-22R1} and between IL-22R1 L2 loop residue, Lys-74^{IL-22R1}, and IL-22 AB loop residues Thr-60^{IL-22} and Asp-61^{IL-22}. Mutation of Lys-74^{IL-22R1} to alanine reduced IL22 functional activity by ˜100-fold, supporting a critical role for this bonding network in IL-22/IL-22R1 interactions (Bleicher et al., FEBS Lett. 582:2985-2992 (2008)).

Consistent with its low affinity and in contrast to IL-10/IL-10R1 and IL-22/IL-22R1, the IL-20/IL-20R1 complex did not form additional site 1a hydrogen bonding networks (FIG. 7D). Lys-74^{IL-22R1}, the key residue in the IL-22/IL-22R1 interface, was replaced by Phe-74^{IL-20R1} and Thr-60^{IL-22} was replaced with Ile-60^{IL-20}. Thus, the IL-20/IL-20R1 site 1a interface consisted of the YG motif and two additional hydrogen bonds (Arg-128^{IL-20R1}/Asp-57^{IL-20} and Glu-105^{IL-20R1}/Arg-63^{IL-20}) that were conserved with IL-22/IL-22R1. Additional disruption of these interactions was predicted in the lower affinity IL-19/IL-20R1 interface where IL-20 residues Ile-60^{IL-20} and Arg63^{IL-20} were replaced by Pro-60^{IL-19} and Thr63^{IL-19} in IL-19.

Mechanisms Regulating IL-20R2 binding to IL-19 and IL-20

Additional SPR experiments revealed IL-19 binds tighter to IL-20R2 (kD=105 nM) than IL-20 (kD=697 nM) (FIGS. 12C and 12D). Superposition of unbound IL-19 onto IL-20 in the ternary complex, revealed most interactions observed in the IL-20/IL-20R2 interface (FIG. 8B, Table 3) were conserved in the IL-19/IL-20R2 complex. However, helix C adopted a different conformation (˜3 [symbol] changes) in IL-19, relative to IL-20 bound to IL-20R2 (FIG. 8C). The conformation of helix C in IL-19 positions Asp-92^{IL-19} into hydrogen bonding distance (3.5 [symbol] ) with the OH of Tyr-78^{IL-20R2}, whereas the equivalent residue in IL-20 (Asp-92^{IL-20}) is 4.3 [symbol] from Tyr-78^{IL-20R2} (FIG. 8D). Replacement of IL-20 Lys-120^{IL-20} with Gln-120^{IL-19} in IL-19 formed an additional hydrogen bond Tyr-78^{IL-20R2} in the IL-19/IL-20R2. The additional hydrogen bonds in the IL-19/IL-20R2 interface were consistent with the higher affinity of this complex than IL-20/IL-20R2 (FIGS. 12C and 12D). The structural differences between IL-19 and IL-20 demonstrated helix C and the CD-loop are conformationally dynamic, which may also influence IL-20R2 binding affinity.

Structural Mechanisms Regulating Cognate Vs. Non-Cognate Ligand Specificity

IL-20R1 and IL-22R1 formed promiscuous interactions with five different cytokines and two R2 chains to engage different signaling responses that protect the host from invading pathogens (FIG. 1). These cognate ligand receptor complexes were “affinity tuned,” using mechanisms described above, to optimize their signaling properties. However, IL-19 cannot bind or signal, through the IL-22R1 chain. Furthermore, IL-22 cannot bind, or induce signaling, through the IL-20R1 chain (FIG. 1). Superposition of IL-22 onto IL-20/IL-20R1 revealed the major specificity determinant was site 1b, where the IL-20R1 L6 loop forms steric clashes with IL-22 helix A residues Phe-57^{IL22} and Asn-54^{IL-22} (FIG. 7F). Similar clashes were observed between IL-22R1 L6 and the N-terminus of IL-19 (FIG. 7E), which explains why IL-19 cannot signal through the type II complex (FIG. 1). Thus, although cognate site 1b contacts were not always extensive, they are clearly critical in determining ligand-receptor specificity. These results provide additional evidence for a two-point (site 1a/site 1b) R1 chain specificity mechanism described by Jones et al., Structure 16:1333-1344 (2008).

Signaling specificity was also achieved through the IL-20R2 chain, which must engage IL-19, IL-20, and IL-24, but prevent IL-22 signaling through the type II (IL-22R1/IL-20R2) complex (FIG. 1) Since IL-22 can signal through the IL-22R1/IL-10R2 heterodimer, but not the IL-22R1/IL-20R2 complex (Dumoutier et al., J. Immunol. 167:3545-3549 (2001)), specificity must occur in the IL-22/IL-20R2 interface. To test this, IL-22 was positioned onto IL-20 in the IL-20 ternary complex. This experiment positioned IL-22 helix C residue, Phe-105^{IL-22}, at the center of the site 2 interface, where it formed extensive steric clashes with IL-20R2 residues Tyr-74^{IL-20R2} and Tyr-78^{IL-20R2} (FIG. 8E). These steric disruptions prevented non-cognate IL-22/IL-20R2 interactions and subsequent cellular signaling by this complex.

The Importance of the Site 3 Interface in Complex Formation and Signaling

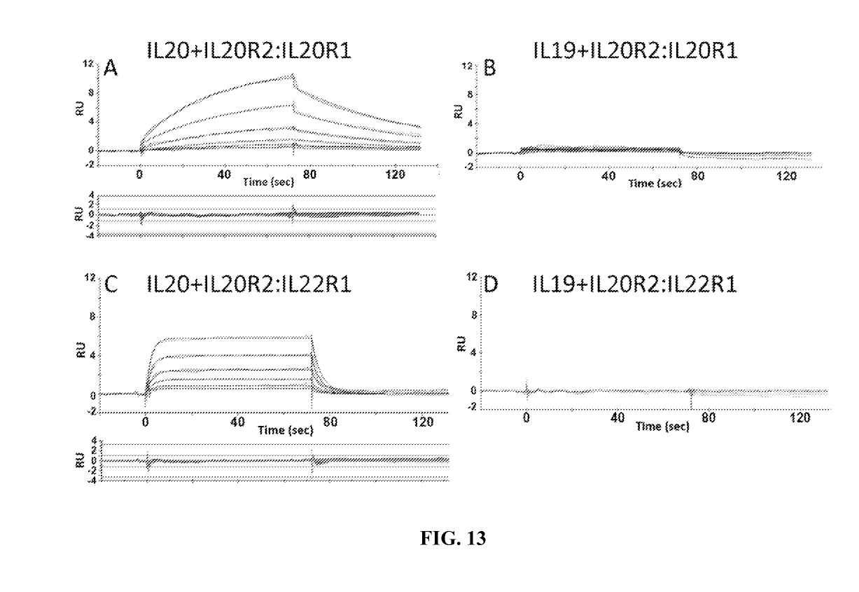

The low affinity of IL-20R1 and IL-20R2 binary complexes emphasized the importance of the site 3 interface (FIGS. 2A and 9A) in forming signaling competent ternary complexes. To estimate the stability of IL-20 (IL-20/IL-20R1/IL-20R2 and IL-20/IL-22R1/IL-20R2) and IL-19 (IL-19/IL-20R1/IL-20R2) ternary complexes (FIG. 1), IL-19, or IL-20, was co-injected with soluble IL-20R1 over a biacore chip coupled with IL-20R2. The apparent affinities obtained from the co-injection experiments (FIGS. 12E-12H) suggested the IL-20 type I complex (FIG. 1) was the most stable complex (KDapp=319 nM), followed by the IL-20 type II complex (KDapp=1,363 nM), and then the IL-19/IL-20R1/IL-20R2 complex (KDapp=3,121 nM). The same results, although with different KDapp values, were obtained by evaluating ternary complex stability by injecting soluble IL-20R2 and IL19, or IL-20, over biacore chip surfaces of IL-20R1 or IL-22R1 (FIG. 13). Kinetic parameters are shown in Table 6 below.

TABLE 6
Kinetic Parameters of Ternary Complex Stability

| Analyte | Ligand | Kon(1/Ms) | Koff (1/s) | KD |
|---|---|---|---|---|
| 20 + 20R2 | IL20R1 | 1.57E+04 | 0.01679 | 1.07 μM |
| 20 + 20R2 | IL22R1 | 2.05E+05 | 0.2379 | 1.16 μM |

To further test the importance of the site 3 interface, three IL-20R1 site 3 residues that form site 3 contacts (FIG. 9A, Ser-190^{IL20R1}, Trp-194^{IL-20R1} and Trp-207^{IL-20R1}) were mutated to alanine to create an IL-20R1 triple mutant, M3. Co-injection of IL-19+M3 over an IL-20R2 biacore surface, generated the same binding response as IL-19 alone (FIG. 9B). Co-injection of IL-20+M3 exhibited an increased binding response, relative to IL-20 alone, although it was drastically reduced from the injection of IL-20+IL-20R1. The increased binding observed for the IL-20+M3 injection corresponds to IL-20/M3 binary complexes binding to IL-20R2 with the same kinetics as the IL-20/IL-20R2 interaction (e.g. without the site 3 interaction). This result confirms the higher affinity of the IL-20/IL-20R1 site 1 interface (FIGS. 12A and 12B), relative to IL-19/IL-20R1, and the importance of site 3 in forming stable ternary complexes essential for signal transduction.

The essential role of the site 3 interface in IL-20 ternary complex formation, led to examination of how the IL-20R1/IL-20R2 D2/D2 interface (site 3) differed from other cytokine ternary complexes. The D2 domains of growth hormone receptor (GHR, site 1, ref de Vos et al., Science 255:306-312 (1992)) and IL-6R (Boulanger et al., Science 300:2101-4 (2003)) were superimposed onto IL-20R1 D2 and the orientations of GHR (site 2) and GP130 D2 domains were evaluated (FIG. 9C). The GHR/GHR D2 domains were essentially parallel to one another and were assigned a D2-D2 crossing angle of 0°. Compared to GHRs, the D2 domains of IL-20R1/IL-20R2 and IL-6R/GP130 D2 cross at angles of −40° and +25°, respectively (FIG. 9C). These differences may be important as the D2 domains are located adjacent to the membrane where they could selectively influence intracellular signal transduction pathways and ultimately cellular responses.

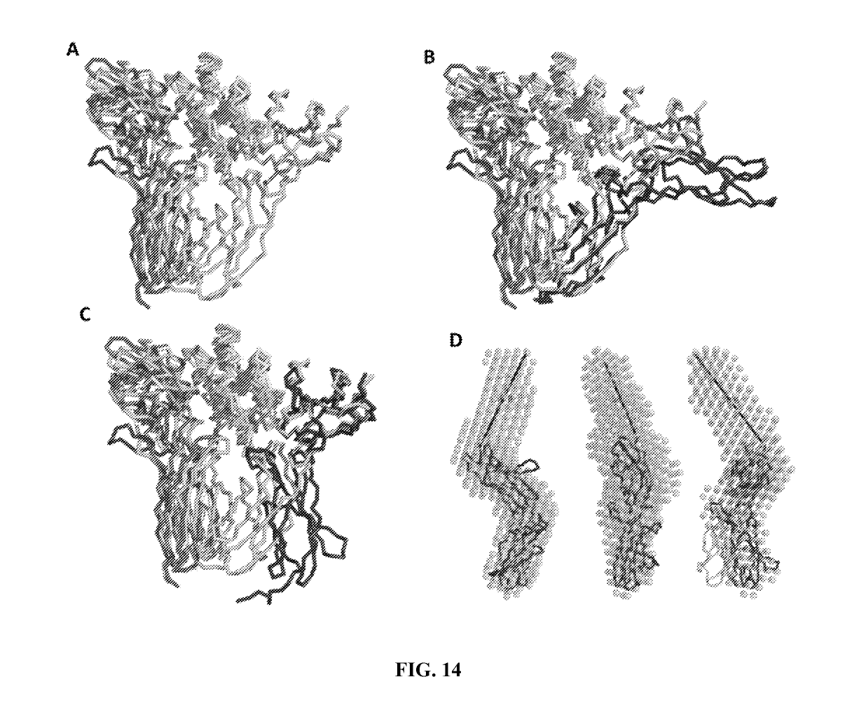

Not only is the IL-20/IL-20R1/IL-20R2 complex distinct from the distantly related class-1 cytokine complexes, but it is also distinct from IL-10R2 containing complexes (FIG. 1). For example superposition of the IL-22/IL-22R1 binary complex and IL-10R2 onto IL-20/IL-20R1/IL-20R2 resulted in an IL-22/IL-22R1/IL-10R2 complex that did not form a site 3 interface (FIG. 9D). This result was caused by the distinct inter-domain angle of IL-20R2 compared to IL-10R2 (FIG. 6). To determine if IL-10R2 adopts a different inter-domain angle in solution, compared to the crystal structure (Yoon et al., Structure 18:638-648 (2010)), solution small angle X-ray scattering (SAXS) was performed on IL-10R2. These experiments confirmed that IL-10R2 in solution adopts the same D1/D2 inter-domain angle observed in the crystal structure (FIG. 14). These studies further underscore the unique architectures of the IL-20R2 and IL-10R2 ternary complexes and their distinct assembly properties.

The crystal structure of IL-20/IL-20R1/IL-20R2 provided herein depicts the first complete signaling complex of an IL-10 family cytokine, although two binary complexes (IL-10/IL-10R1, IL-22/IL-22R1) have been determined (Josephson et al., Immunity 15:33-46 (2001); Jones et al., Structure 16:1333-44 (2008); and Bleicher et al., FEBS Lett. 582:2985-2992 (2008)). The surprising structural differences between IL-19 and IL-20 at the N- and C-termini (FIG. 5) explain how structurally similar cytokines discriminate, via IL-20R1 and IL-22R1, between the type I and type II receptor heterodimers (FIGS. 8E and 8F). In contrast, IL-22, which binds tightly to IL-22R1, cannot signal through the type II complex due to steric clashes between IL-22/IL-20R2 in site 2, especially with Phe-105^{IL22} (FIG. 9E). These results provide a structural basis for IL-20 SFC receptor specificity, which contributes to their distinct in-vivo biological properties.

In addition to discriminating between the type I and type II complexes (FIG. 1), IL-19 and IL-20 modulate their biological activities through distinct affinities for IL-20R1 and IL-20R2 chains (FIG. 12). The structural studies herein provide a molecular basis for how subtle structural rearrangements of the receptor interfaces, combined with amino acid substitutions, alter ligand receptor binding affinity. Prior experiments revealed IL-20R2 binds tighter to IL-19 and IL-20 than IL-20R1 (Parrish-Novak et al., J. Biol. Chem. 277:47517-47523 (2002); Pletnev, et al., Biochemistry 42:12617-12624 (2003)). However, in contrast to ˜1 nM IL-10/IL-10R1 and IL-22/IL-22R1 affinities (Table 5), IL-19/IL-20R2 and IL-20/IL-20R2 interactions were at least ˜100-fold weaker. Thus, in contrast to IL-10/IL-10R1 and IL-22/IL-22R1 complexes (FIG. 1), the IL-20R2 chain did not dominate ligand binding energetics, but relies on cooperation between IL-20R1 and IL-20R2 to assemble the signaling complex. This property allows the type-I receptor heterodimer (FIG. 1) to selectively discriminate IL-19, IL-20, and IL-24 affinity differences and induce distinct cellular responses. Using SPR (FIG. 6), the stability of the IL-20 type-I and type-II complexes were found to be more stable than the IL-19 type-I complex (FIGS. 1 and 12). The molecular stabilities of the ternary complexes (IL-20>IL-19) were consistent with robust IL-20 signaling in keratinocytes and its putative role in psoriasis; properties not shared by IL-19 (Blumberg et al., Cell 104:9-19 (2001); Parrish-Novak et al., J. Biol. Chem. 277:47517-47523 (2002)).

The presence of an IL-20/IL-20R1/IL-20R2 dimer in the crystals (FIG. 2D) provided a possible explanation for how IL-20 SFCs might signal on immune cells in the absence of IL-20R1 (Gallagher, Cytokine Growth Factor Rev. 21:345-352 (2010); Wahl et al., J. Immunol. 182:802-810 (2009); Nagalakshmi et al., Int. Immunopharmacol. 4:577-592 (2004)). However, biochemical evidence was not able to be generated to confirm IL-19 or IL-20, can dimerize IL-20R2 in the absence of IL-20R1 (FIG. 11). Our studies show the main signaling unit of IL-19 and IL-20 were the ternary complexes, which may further oligomerize on cells.

Protein Expression and Purification of the Complex

IL-20 (residues 25-176, uniprot Q9NYY1) was expressed with an N-terminal histidine tag in insect cells. IL-20R1 (residues 29-245, uniprot Q9UHF4), and IL-20R2 (residues 30-231, uniprot Q6UXL0) were expressed insect cells with C-terminal histidine tags. Two N-linked glycosylation attachment sites in IL-20R2, (Asn-40^{IL-20R2} and Asn-134^{IL-20R2}) were removed by mutagenesis (QuikChange™, Stratagene, La Jolla, Calif.) converting the asparagines to glutamines to yield IL-20R2^{QQ}, used for crystallization studies. IL-20R1 was modified by mutation of Lys-111^{IL-20R1} and Lys-113^{IL-20R1} to arginines (IL-20R1^{RR}) for crystallization. IL-20, and the receptors, were purified by nickel affinity chromatography. The histidine tags of all three proteins were removed by incubation with FactorXa protease. The individual proteins were incubated at approximately a 1:1:1 stoichiometric ratio and purified by gel filtration chromatography. Fractions containing the ternary complex were concentrated to 7 mg/mL for crystallization.

Crystallization, Data Collection, and Structure Determination

A partial model of IL-20/IL-20R1/IL-20R2 was obtained by SAD phasing weakly diffracting tetragonal crystals (Logsdon et al., Acta Crystallogr. Sect. F. Struct. Biol. Cryst. Commun. 68:89-92 (2012). Higher quality diffraction data (2.8 [symbol] resolution) was obtained by crystallizing the complex in a different spacegroup (P2₁2₁2₁). This was achieved by purifying the complex containing an IL-20R1 mutant, where Lys-111^{IL-20R1} and Lys-113^{IL-20R1} were mutated to arginine (IL-20R1^{RR}). Crystals of IL-20/IL-20R1^{RR}/IL-20R2^{QQ} were grown by hanging-drop vapor diffusion from solutions of 12% PEG 6000, 0.1 M ADA, pH 6.5, and 0.1M MgCl₂. The crystals were cryoprotected for data collection at 100° K in a solution of 19% PEG 6000, 0.1 M ADA, pH 6.5, 0.1M MgCl₂ and 15% glycerol. The diffraction quality of the best crystal was ˜4.6 [symbol] resolution on a rotating anode x-ray source that was improved to 3.3 [symbol] resolution by crystal annealing. A complete dataset extending to 2.8 [symbol] resolution was collected at the Advance Photon Source, SER-CAT (Table 2). The data were processed using HKL2000 (Otwinowski & Minor W., eds. Processing x-ray diffraction data collected in oscillation mode Academic Press, Pasadena, Calif. (1997)).

The partial model was positioned and refined against the IL-20/IL-20R1^{RR}/IL-20R2^{QQ} data using Phenix (Adams et al., Acta Crystallogr. D. Biol. Crystallogr. 54(Pt.5):905-921 (1998)). Refinement, that included carbohydrate, was performed using CNS 1.1 (Brunger et al., Acta Crystallogr. D. Biol. Crystallogr. 54(Pt.5):905-921 (1998)). The final model was refined at 2.8 [symbol] resolution to R_{cryst} and R_{free} values of 23.0% and 27.8%, respectively (Table 2). The crystals contain two IL-20/IL-20R1/IL-20R2 ternary complexes in the asymmetric unit, which are essentially identical (r.m.s.d.=0.4 [symbol] ). The final model contains IL-20 residues 24-176, IL-20R1 residues 24-227, and IL-20R2 residues 34-226. N-linked carbohydrate was observed attached to Asn-27 and Asn-167 of IL-20R1. Model evaluation and minor rebuilding was performed using O (Jones et al., Acta Crystallogr. D. Biol. Crystallogr. 47:283-290 (1991)). Buried surface area was calculated using NACCESS (Hubbard S J & Thorton J M (1993) NACCESS Computer Program, Depart. Biochem., Molec. Biol., University College London). Structural alignments were performed using STAMP (Russell and Barton, Proteins 14:309-323 (1992) implemented in V M D Humphrey et al., J. Mol. Graph. 14(1):33-8, 27-8 (1996)). Figures were made using Pymol (DeLano W L (2002) The PyMOL Molecular Graphics System DeLano Scientific, San Carlos, Calif., USA).

Surface Plasmon Resonance Experiments SPR experiments were performed on Biacore T-100, and T-200, (GE healthcare, Waukesha, Wis.) systems. Soluble IL-20R2 was amine coupled (100-130 RU) to CM5 research grade biacore chips in 10 mM sodium acetate, pH 5.0. IL-20R1-FC and IL-20R2-FC were coupled (60-120 RU) to CM5 chips using an anti-FC Ab coupling kit using the manufacturer's instructions (GE healthcare, Waukesha, Wis.). All binding experiments were performed in running buffer consisting of 10 mM Hepes, pH 7.4, 150 mM NaCl, 0.04% P20, and 0.1 mg/mL bovine serum albumin (Sigma, St. Louis, Mo.). No regeneration was performed between sample injections on IL-20R2 amine-coupled surfaces. IL-20R1-FC and IL-20R2-FC surfaces were regenerated after each sample injection with a 3-minute injection of glycine, pH 1.7. Soluble proteins were injected for 75-90 seconds (30 uL/min) over the surfaces, and the dissociation phase was followed for 75-120 seconds depending on the stability of the complex being analyzed. Where possible, the resultant sensorgrams were fit to 1:1 binding models using Biacore T-200 evaluation software version 1. Binding parameters obtained from these experiments are shown in Table 4.

IL-19/IL-20 Binding to Amine Coupled and FC-Coupled IL-20R2 Surfaces

IL-19/IL-20R2 and IL-20/IL-20R2 interactions were determined by SPR methods with IL-20R2 attached to a biacore chip by amine coupling, or as an IL-20R2-FC fusion protein. Amine coupled IL-20R2 should not be able to form IL-20R2 dimers, whereas the IL-20R2-FC protein should be able to assemble the dimer. If dimerization occurs, it should result in an increase in analyte (IL19 or IL-20) affinity.

The results of the experiments are shown in Table 4. For IL-19/IL-20R2, complex stability was measured by comparing off-rates of IL-19/IL-20R2 interactions on the two surfaces. This analysis revealed the IL19/IL-20R2-FC interaction is ˜60% more stable (koff=0.0242 sec⁻¹) than the IL-19 binding to amine coupled IL-20R2 (koff=0.0398 sec⁻¹). The data quality of IL-20 binding to amine coupled IL-20R2 was not of high quality and kinetic rate constants could not be obtained. Comparison of equilibrium affinity constants revealed IL-20/IL-20-FC binding was ˜30% tighter than IL-20 binding to amine coupled IL-20R2. Thus, IL-19/IL-20R2 and IL-20/IL-20R2 complex stability did increase modestly upon binding the IL-20R2-FC. However, the differences were not significant given the different methods used to couple IL-20R2.

Cross Linking IL-20R2 Complexes

To further test IL-20R2 dimer formation, cross-linking experiments were performed on IL-19/IL-20R2, IL-19/IL-20R1/IL-20R2, and IL-20/IL-20R1/IL-20R2 soluble complexes (FIG. 11). Glutaraldehyde (EM sciences, concentrations of 0.005%, 0.01%, 0.02%, and 0.05%) cross linking was performed on each complex at a concentration of 5 μM for 30 minutes. Cross-linked complexes were separated on 15% SDS-PAGE gels. The gels reveal IL-19/IL-20R2 forms a stable 1:1 complex, but did not form IL-19/IL-20R2 dimers (FIG. 11A). In experiments performed with IL-19/IL-20R1/IL-20R2 (FIG. 11B), the predominant species remained the IL-19/IL-20R2 binary complex, although a light band with a molecular weight corresponding to an IL-19/IL-20R1/IL-20R2 dimer was also observed. A similar result was observed upon cross-linking IL-20/IL-20R1/IL-20R2, however the intense band for corresponding to the IL-20/IL-20R2 binary complex was not observed (FIG. 11C).

Taken together, the SPR (Table 4) and cross-linking studies (FIG. 11) suggest IL-19 and IL-20 cannot assemble soluble IL-19/IL-20R2, or IL-20/IL-20R2, dimer complexes in vitro. This suggested dimer complexes will not be formed on the cell surface, although the influence of the intracellular domains on IL-20R2 dimerization has not been addressed. Weak bands corresponding to IL-19 and IL-20 ternary complex dimers were observed by cross-linking. However, this appears to be very inefficient and/or transient as the intensity of the band, relative to the total amount of protein in the experiment was very small. In addition, dimeric complexes were not observed during purification of the ternary complex for crystallization. Thus, biochemical analysis of the IL-20 dimer complex suggested it did not play a significant role in IL-19 and IL-20 signaling. Rather, the main signaling unit of IL-19 and IL-20 was likely the 1:1:1 ternary complex.

Small Angle X-Ray Scattering Analysis

X-ray scattering data on IL-10R2, containing an N-terminal histidine tag (10 mg/mL) was collected on the SIBYLS beamline at the Advanced Light Source (ALS), Berkley. Scattering curves were processed with the program PRIMUS within the ATSAS package (Konarev et al., J. Appl. Cryst. 36:1277-1282 (2003)). Bead models were derived from the scattering curve with the program DAMN (Svergun, Biophys. J. 76:2879-2886 (1999)).

Modeling IL-10R2 Containing Cytokine Complexes

It was determined if the IL-20 ternary complex could serve as a structural scaffold to model receptor complexes containing the IL-10R2 chain (e.g. IL-22/IL-22R1/IL-10R2 and IL-10/IL-10R1/IL-10R2). To address this question, IL-22/IL-22R1 and IL-10/IL-10R1 binary complexes were superimposed onto the IL-20/IL-20R1/IL-20R2 complex (FIG. 13A). In this superposition, the position of IL-20R2 provided a reasonable location for the IL-10R2 chain. However, superimposing IL-10R2 D2 domain onto IL-20R2 (FIG. 13B) positions IL-10R2 such that the D1 domain did not contact IL-22 or IL-10, which was inconsistent with prior biochemical studies (Yoon et al., Structure 18:638-648 (2010); Logsdon et al., J. Mol. Biol. 342:503-514 (2004); Yoon et al., J. Biol. Chem. 281:35088-35096 (2006); and Wu et al., J. Mol. Biol. 382:1168-1183 (2008)). Superposition of the IL-10R2 D1 domain onto IL-20R2 (FIG. 13C) positions IL-10R2 D1 for interactions with IL-10 and IL-22. However, in this model, the D2 domains of the IL-22R1/IL-10R2 and IL-10R1/IL-10R2 were separated by ˜15 [symbol] (FIG. 13C) and did not form a site 3 interface with IL-22R1 and IL-10R1.

The gap between the receptors might be closed by a change in the inter-domain angle of IL-10R2. To test this possibility, small angle x-ray scattering (SAXS) was performed on the IL-10R2 chain. DAMIN bead models derived from the scattering data were consistent with the inter-domain angle observed in the IL-10R2 crystal structure (FIG. 13D). This suggested that IL-10R2 either undergoes a rigid-body rotation to form site 3 interfaces with IL22R1 and IL-10R1, or IL-10R2 did not form site 3 interfaces with IL-10/IL-10R1 and IL-22/IL-22R1 binary complexes. The later hypothesis was consistent with the extremely low affinity of IL-10R2 for its binary complexes (Yoon et al., Structure 18:638-648 (2010); Logsdon et al., J. Mol. Biol. 342:503-514 (2004); and Yoon et al., Structure 13:551-564 (2005)). A D2-D2 membrane proximal interaction was not observed in the recent structure of the IFNα/IFNAR1/IFNAR2 ternary complex (Thomas et al., Cell 146:621-632 (2011)), which further indicated that IL-10R2 contacts with R1 chain D2 domains very transiently, or do not occur at all.

Sequences

IL-19 (SEQ ID NO: 1)
MASVDNHGLRRCLISTDMHHIEESFQEIKRAIQAKDTFPNVTILSTLETLQIIKPL
DVCCVTKNLLAFYVDRVFKDHQEPNPKILRKISSIANSFLYMQKTLRQCQEQRQCHCR
QEATNATRVIHDNYDQLEVHAAAIKSLGELDVFLAWINKNHEVMFSA
IL-19 cDNA (SEQ ID NO: 2)
atggcctcagtagacaaccacggtctcaggagatgtctgatttccacagacatgcaccatatagaagagagtttccaagaaat
caaaagagccatccaagctaaggacaccttcccaaatgtcactatcctgtccacattggagactctgcagatcattaagcccttagatgtgt
gctgcgtgaccaagaacctcctggcgttctacgtggacagggtgttcaaggatcatcaggagccaaaccccaaaatcttgagaaaaatc
agcagcattgccaactctttcctctacatgcagaaaactctgcggcaatgtcaagaacagaggcagtgtcactgcaggcaggaagccac
caatgccaccagagtcatccatgacaactatgatcagctggaggtccacgctgctgccattaaatccctgggagagctcgacgtctttcta
gcctggattaataagaatcatgaagtaatgttctcagcttga
IL-20 (SEQ ID NO: 3)
MLKTLNLGSCVIATNLQEIRNGFSEIRGSVQAKDGNIDIRILRRTESLQDTKPANR
CCLLRHLLRLYLDRVFKNYQTPDHYTLRKISSLANSFLTIKKDLRLCHAHMTCHCGEE
AMKKYSQILSHFEKLEPQAAVVKALGELDILLQWMEETE
IL-20 cDNA (SEQ ID NO: 4)
atgctgaagacactcaatttgggaagctgtgtgatcgccacaaaccttcaggaaatacgaaatggattttctgagatacggggc
agtgtgcaagccaaagatggaaacattgacatcagaatcttaaggaggactgagtctttgcaagacacaaagcctgcgaatcgatgctg
cctcctgcgccatttgctaagactctatctggacagggtatttaaaaactaccagacccctgaccattatactctccggaagatcagcagcc
tcgccaattcctttcttaccatcaagaaggacctccggctctgtcatgcccacatgacatgccattgtggggaggaagcaatgaagaaata
cagccagattctgagtcactttgaaaagctggaacctcaggcagcagttgtgaaggctttgggggaactagacattatctgcaatggatg
gaggagacagaatag
IL-20R1 (SEQ ID NO: 5)
VPCVSGGLPKPANITFLSINMKNVLQWTPPEGLQGVKVTYTVQYFIYGQKKWLNKSE
CRNINRTYCDLSAETSDYEHQYYAKVKAIWGTKCSKWAESGRFYPFLETQIGPPEVALT
TDEKSISVVLTAPEKWKRNPEDLPVSMQQIYSNLKYNVSVLNTKSNRTWSQCVTNHTL
VLTWLEPNTLYCVHVESFVPGPPRRAQPSEKQCARTLKDQSS
IL-20R1 cDNA (SEQ ID NO: 6)
gttccctgtgtctctggtggtttgcctaaacctgcaaacatcaccttcttatccatcaacatgaagaatgtcctacaatggactcca
ccagagggtcttcaaggagttaaagttacttacactgtgcagtatttcatatatgggcaaaagaaatggctgaataaatcagaatgcagaaa
tatcaatagaacctactgtgatctttctgctgaaacttctgactacgaacaccagtattatgccaaagttaaggccatttggggaacaaagtg
ttccaaatgggctgaaagtggacggttctatccttttttagaaacacaaattggcccaccagaggtggcactgactacagatgagaagtcc
atttctgttgtcctgacagctccagagaagtggaagagaaatccagaagaccttcctgtttccatgcaacaaatatactccaatctgaagtat
aacgtgtctgtgttgaatactaaatcaaacagaacgtggtcccagtgtgtgaccaaccacacgctggtgctcacctggctggagccgaac
actctttactgcgtacacgtggagtccttcgtcccagggccccctcgccgtgctcagccttctgagaagcagtgtgccaggactttgaaag
atcaatcatcaattgaaggtagactcgagtctagagggcccttcgaaggtaagcctatccctaaccctctcctcggtctcgattctacgcgt
accggtcatcatcaccatcaccattga
IL-20R2 (SEQ ID NO: 7)
DEVAILPAPQQLSVLSTNMKHLLMWSPVIAPGETVYYSVEYQGEYESLYTSHIW
IPSSWCSLTEGPECDVTDDITATVPYNLRVRATLGSQTSAWSILKHPFNRQSTILTRPGME
ITKDGFHLVIELEDLGPQFEFLVAYWRREPGAEEHVKMVRSGGIPVHLETMEPGAAYC
VKAQTFVKAIGRYSAFSQTECVEVQGEA
IL-20R2 cDNA (SEQ ID NO: 8)
gatgaagtggccattctgcctgcccctcagcagctctctgtactctcaaccaacatgaagcatctcttgatgtggagcccagtg
atcgcgcctggagaaacagtgtactattctgtcgaataccagggggagtacgagagcctgtacacgagccacatctggatccccagca
gctggtgctcactcactgaaggtcctgagtgtgatgtcactgatgacatcacggccactgtgccatacaaccttcgtgtcagggccacatt
gggctcacagacctcagcctggagcatcctgaagcatccctttaatagaaactcaaccatccttacccgacctgggatggagatcaccaa
agatggcttccacctggttattgagctggaggacctggggccccagtttgagttccttgtggcctactggaggagggagcctggtgccga
ggaacatgtcaaaatggtgaggagtgggggtattccagtgcacctagaaaccatggagccaggggctgcatactgtgtgaaggcccag
acattcgtgaaggccattgggaggtacagcgccttcagccagacagaatgtgtggaggtgcaaggagaggccattgaaggtagactcg
agtctagagggcccttcgaaggtaagcctatccctaaccctctcctcggtctcgattctacgcgtaccggtcatcatcaccatcaccattga
IL-22R1 DDQ (SEQ ID NO: 9)
HAPEDPSDLLQHVKFQSSNFENILTWDSGPEGTPDTVYSIEYKTYGERDWVAK
KGCQRITRKSCDLTVETGDLQELYYARVTAVSAGGRSATKMTDRFSSLQHTTLKPPDVT
CISKVRSIQMIVHPTPTPIRAGDGHRLTLEDIFHDLFYHLELQVNRTYQMHLGGKQREY
EFFGLTPDTEFLGTIMICVPTWAKESAPYMCRVKTLPDRT
IL-22R1 DDQ cDNA (SEQ ID NO: 10)
cacgcccctgaggacccctcggatctgctccagcacgtgaaattccagtccagcaactttgaaaacatcctgacgtgggaca
gcgggccagagggcaccccagacacggtctacagcatcgagtataagacgtacggagagagggactgggtggcaaagaagggctg
tcagcggatcacccggaagtcctgcgacctgacggtggagacgggcgacctccaggagctctactatgccagggtcaccgctgtcagt
gcgggaggccggtcagccaccaagatgactgacaggttcagctctctgcagcacactaccctcaagccacctgatgtgacctgtatctc
caaagtgagatcgattcagatgattgttcatcctacccccacgccaatccgtgcaggcgatggccaccggctaaccctggaagacatctt
ccatgacctgttctaccacttagagctccaggtcaaccgcacctaccaaatgcaccttggagggaagcagagagaatatgagttcttcgg
cctgacccctgacacagagttccttggcaccatcatgatttgcgttcccacctgggccaaggagagtgccccctacatgtgccgagtgaa
gacactgccagaccggacaattgaaggtagagtcgagtctagagggcccttcgaaggtaagcctatccctaaccctctcctcggtctcg
attctacgcgtaccggtcatcatcaccatcaccattga
IL-20R1 wt FCk (SEQ ID NO: 11)
VPCVSGGLPKPANITFLSINMKNVLQWTPPEGLQGVKVTYTVQYFIYGQKKWL
NKSECRNINRTYCDLSAETSDYEHQYYAKVKAIWGTKCSKWAESGRFYPFLETQIGPP
EVALTTDEKSISVVLTAPEKWKRNPEDLPVSMQQIYSNLKYNVSVLNTKSNRTWSQCV
TNHTLVLTWLEPNTLYCVHVESFVPGPPRRAQPSEKQCARTLKDQSSEFKAKGSGGGG
PSVFIFPPKIKDVLMISLSPIVTCVVVDVSEDDPDVQISWFVNNVEVHTAQTQTHREDY
NSTLRVVSALPIQHQDWMSGKEFKCKVNNKDLPAPIERTISKPKGSVRAPQVYVLPPPC
EEMTKKQVTLYCMVTDFMPEDIYVEWTNNGKTELNYKNTEPVLDSDGSYFMYSKLR
VEKKNWVERNSYSCSVVHEGLHNHHTTKSFSRTPGKGLEASGGLNDIFEAQKIEWHE
GTGHHHHHH
IL-20R1 wt FCk cDNA (SEQ ID NO: 12)
gttccctgtgtctctggtggtttgcctaaacctgcaaacatcaccttcttatccatcaacatgaagaatgtcctacaatggactcca
ccagagggtcttcaaggagttaaagttacttacactgtgcagtatttcatatatgggcaaaagaaatggctgaataaatcagaatgcagaaa
tatcaatagaacctactgtgatctttctgctgaaacttctgactacgaacaccagtattatgccaaagttaaggccatttggggaacaaagtg
ttccaaatgggctgaaagtggacggttctatccttttttagaaacacaaattggcccaccagaggtggcactgactacagatgagaagtcc
atttctgttgtcctgacagctccagagaagtggaagagaaatccagaagaccttcctgtttccatgcaacaaatatactccaatctgaagtat
aacgtgtctgtgttgaatactaaatcaaacagaacgtggtcccagtgtgtgaccaaccacacgctggtgctcacctggctggagccgaac
actctttactgcgtacacgtggagtccttcgtcccagggccccctcgccgtgctcagccttctgagaagcagtgtgccaggactttgaaag
atcaatcatcagagtttaaagccaaaggatccggaggaggtggtccatccgtcttcatcttccctccaaagatcaaggatgtactcatgatc
tccctgagccccatagtcacatgtgtggtggtggatgtgagcgaggatgacccagatgtccagatcagctggtttgtgaacaacgtggaa
gtacacacagctcagacacaaacccatagagaggattacaacagtactctccgggtggtcagtgccctccccatccagcaccaggact
ggatgagtggcaaggagttcaaatgcaaggtcaacaacaaagacctcccagcgcccatcgagagaaccatctcaaaacccaaagggt
cagtaagagctccacaggtatatgtcttgcctccaccatgcgaagagatgactaagaaacaggtcactctgtactgcatggtcacagactt
catgcctgaagacatttacgtggagtggaccaacaacgggaaaacagagctaaactacaagaacactgaaccagtcctggactctgatg
gttcttacttcatgtacagcaagctgagagtggaaaagaagaactgggtggaaagaaatagctactcctgttcagtggtccacgagggtct
gcacaatcaccacacgactaagagcttctcccggactccgggtaaaggtctcgaggctagcggaggactcaacgacatcttcgaagca
caaaaaatcgaatggcacgaaggtaccggtcatcatcaccatcaccattga
IL-22R1 FCk (SEQ ID NO: 13)
AHAPEDPSDLLQHVKFQSSNFENILTWDSGPEGTPDTVYSIEYKTYGERDWVA
KKGCQRITRKSCDLTVETGDLQELYYARVTAVSAGGRSATKMTDRFSSLQHTTLKPPD
VTCISKVRSIQMIVHPTPTPIRAGDGHRLTLEDIFHDLFYHLELQVNRTYQMHLGGKQR
EYEFFGLTPDTEFLGTIMICVPTWAKESAPYMCRVKTLPDRTWTYSGSGGGGPSVFIFPP
KIKDVLMISLSPIVTCVVVDVSEDDPDVQISWFVNNVEVHTAQTQTHREDYNSTLRVV
SALPIQHQDWMSGKEFKCKVNNKDLPAPIERTISKPKGSVRAPQVYVLPPPCEEMTKK
QVTLYCMVTDFMPEDIYVEWTNNGKTELNYKNTEPVLDSDGSYFMYSKLRVEKKNW
VERNSYSCSVVHEGLHNHHTTKSFSRTPGKGLEASGGLNDIFEAQKIEWHEGTGHHHH
HH
IL-22R1 FCk-His-4G cDNA (SEQ ID NO: 14)
gctcacgcccctgaggacccctcggatctgctccagcacgtgaaattccagtccagcaactttgaaaacatcctgacgtggg
acagcgggccagagggcaccccagacacggtctacagcatcgagtataagacgtacggagagagggactgggtggcaaagaaggg
ctgtcagcggatcacccggaagtcctgcgacctgacggtggagacgggcgacctccaggagctctactatgccagggtcaccgctgtc
agtgcgggaggccggtcagccaccaagatgactgacaggttcagctctctgcagcacactaccctcaagccacctgatgtgacctgtat
ctccaaagtgagatcgattcagatgattgttcatcctacccccacgccaatccgtgcaggcgatggccaccggctaaccctggaagacat
cttccatgacctgttctaccacttagagctccaggtcaaccgcacctaccaaatgcaccttggagggaagcagagagaatatgagttcttc
ggcctgacccctgacacagagttccttggcaccatcatgatttgcgttcccacctgggccaaggagagtgccccctacatgtgccgagtg
aagacactgccagaccggacatggacctactccggatccggaggaggtggtccatccgtcttcatcttccctccaaagatcaaggatgta
ctcatgatctccctgagccccatagtcacatgtgtggtggtggatgtgagcgaggatgacccagatgtccagatcagctggtttgtgaaca
acgtggaagtacacacagctcagacacaaacccatagagaggattacaacagtactctccgggtggtcagtgccctccccatccagca
ccaggactggatgagtggcaaggagttcaaatgcaaggtcaacaacaaagacctcccagcgcccatcgagagaaccatctcaaaacc
caaagggtcagtaagagctccacaggtatatgtcttgcctccaccatgcgaagagatgactaagaaacaggtcactctgtactgcatggt
cacagacttcatgcctgaagacatttacgtggagtggaccaacaacgggaaaacagagctaaactacaagaacactgaaccagtcctg
gactctgatggttcttacttcatgtacagcaagctgagagtggaaaagaagaactgggtggaaagaaatagctactcctgttcagtggtcc
acgagggtctgcacaatcaccacacgactaagagcttctcccggactccgggtaaaggtctcgaggctagcggaggactcaacgacat
cttcgaagcacaaaaaatcgaatggcacgaaggtaccggtcatcatcaccatcaccattga
IL-20R2 FCh (SEQ ID NO: 15)
DEVAILPAPQQLSVLSTNMKHLLMWSPVIAPGETVYYSVEYQGEYESLYTSHIW
IPSSWCSLTEGPECDVTDDITATVPYNLRVRATLGSQTSAWSILKHPFNRQSTILTRPGME
ITKDGFHLVIELEDLGPQFEFLVAYWRREPGAEEHVKMVRSGGIPVHLETMEPGAAYC
VKAQTFVKAIGRYSAFSQTECVEVQGEAGSGGGGPSVFIFPPKIKDVLMISLSPIVTCVV
VDVSEDDPDVQISWFVNNVEVHTAQTQTHREDYNSTLRVVSALPIQHQDWMSGKEFK
CKVNNKDLPAPIERTISKPKGSVRAPQVYVLPPPEEEMTKKQVTLTCMVTDFMPEDIYV
EWTNNGKTELNYKNTEPVLDSDGSYFMTSKLRVEKKNWVERNSYSCSVVHEGLHNH
HTTKSFGGCGTPASGGLNDIFEAQKIEWHE
IL-20R2 FCh cDNA (SEQ ID NO: 16)
gatgaagtggccattctgcctgcccctcagcagctctctgtactctcaaccaacatgaagcatctcttgatgtggagcccagtg
atcgcgcctggagaaacagtgtactattctgtcgaataccagggggagtacgagagcctgtacacgagccacatctggattcccagcag
ctggtgctcactcactgaaggtcctgagtgtgatgtcactgatgacatcacggccactgtgccatacaaccttcgtgtcagggccacattg
ggctcacagacctcagcctggagcatcctgaagcatccctttaatagacagtcaaccatccttacccgacctgggatggagatcaccaaa
gatggcttccacctggttattgagctggaggacctggggccccagtttgagttccttgtggcctactggaggagggagcctggtgccgag
gaacatgtcaaaatggtgaggagtgggggtattccagtgcacctagaaaccatggagccaggggctgcatactgtgtgaaggcccaga
cattcgtgaaggccattgggaggtacagcgccttcagccagacagaatgtgtggaggtgcaaggagaggccggatccggaggaggt
ggtccatccgtcttcatcttccctccaaagatcaaggatgtactcatgatctccctgagccccatagtcacatgtgtggtggtggatgtgagc
gaggatgacccagatgtccagatcagctggtttgtgaacaacgtggaagtacacacagctcagacacaaacccatagagaggattaca
acagtactctccgggtggtcagtgccctccccatccagcaccaggactggatgagtggcaaggagttcaaatgcaaggtcaacaacaa
agacctcccagcgcccatcgagagaaccatctcaaaacccaaagggtcagtaagagctccacaggtatatgtcttgcctccaccagaag
aagagatgactaagaaacaggtcactctgacctgcatggtcacagacttcatgcctgaagacatttacgtggagtggaccaacaacggg
aaaacagagctaaactacaagaacactgaaccagtcctggactctgatggttcttacttcatgaccagcaagctgagagtggaaaagaa
gaactgggtggaaagaaatagctactcctgttcagtggtccacgagggtctgcacaatcaccacacgactaagagcttcggaggttgcg
gaacaccagctagcggaggactcaacgacatcttcgaagcacaaaaaatcgaatggcacgaatga
IL-24 (SEQ ID NO: 17)
AQGQEFHFGPCQVKGVVPQKLWEAFWAVKDTMQAQDNITSARLLQQEVLQN
VSDAESCYLVHTLLEFYLKTVFKNYHNRTVEVRTLKSFSTLANNFVLIVSQLQPSQENE
MFSIRDSAHRRFLLFRRAFKQLDVEAALTKALGEVDILLTWMQKFYKL
IL-24 cDNA (SEQ ID NO: 18)
gcccagggccaagaattccactttgggccctgccaagtgaagggggttgttccccagaaactgtgggaagccttctgggctg
tgaaagacactatgcaagctcaggataacatcacgagtgcccggctgctgcagcaggaggttctgcagaacgtctcggatgctgagag
ctgttaccttgtccacaccctgctggagttctacttgaaaactgttttcaaaaactaccacaatagaacagttgaagtcaggactctgaagtc
attctctactctggccaacaactttgttctcatcgtgtcacaactgcaacccagtcaagaaaatgagatgttttccatcagagacagtgcaca
caggcggttcctgctattccggagagcattcaaacagttggacgtagaagcagctctgaccaaagcccttggggaagtggacattcttct
gacctggatgcagaaattctacaagctctga
IL-20 M1 (SEQ ID NO: 19)
SSMKTLNLGSCVIATNLQEIRNGFSEIRGSVQAKDGNIDIRILRRTESLQDTKPAN
RCCLLRHLLRLYLDRVFKNYQTPDHYTLRKISSLANSFLTIKKDLRLCHAHMTCHCGE
EAMKKYSQILSHFEKLEPQAAVVKALGELDILLQWMEETE
IL-20 M2 (SEQ ID NO: 20)
CSSMKTLNLGSCVIATNLCEIRNGFSEIRGSVQAKDGNIDIRILRRTESLQDTKPA
NRCCLLRHLLRLYLDRVFKNYQTPDHYTLRKISSLANSFLTIKKDLRLCHAHMTCHCG
EEAMKKYSQILSHFEKLEPQAAVVKALGELDILLQWMEETE
IL-19/IL-20 Hybrid (SEQ ID NO: 21)
MNHGLRRCLISTDLQEIRNGFSEIRGSVQAKDGNIDIRILRRTESLQDTKPANRC
CLLRHLLRLYLDRVFKNYQTPDHYTLRKISSLANSFLTIKKDLRLCHAHMTCHCGEEA
MKKYSQILSHFEKLEPQAAVVKALGELDILLQWMEEHEVMFSA*
IL-19/IL-20 Hybrid cDNA (SEQ ID NO: 22)
atgaaccacggtctcaggagatgtctgatttccacagaccttcaggaaatacgaaatggattttctgagatacggggcagtgtg
caagccaaagatggaaacattgacatcagaatcttaaggaggactgagtctttgcaagacacaaagcctgcgaatcgatgctgcctcctg
cgccatttgctaagactctatctggacagggtatttaaaaactaccagacccctgaccattatactctccggaagatcagcagcctcgcca
attcctttcttaccatcaagaaggacctccggctctgtcatgcccacatgacatgccattgtggggaggaagcaatgaagaaatacagcca
gattctgagtcactttgaaaagctggaacctcaggcagcagttgtgaaggctttgggggaactagacattcttctgcaatggatggaggag
catgaagtaatgttctcagcttag
IL-22 wt ( SEQ ID NO: 23)
PISSHCRLDKSNFQQPYITNRTFMLAKEASLADNNTDVRLIGEKLFHGVSMSER
CYLMKQVLNFTLEEVLFPQSDRFQPYMQEVVPFLARLSNRLSTCHIEGDDLHIQRNVQ
KLKDTVKKLGESGE IKAIGELDLLFMSLRNACI
IL-22 wt cDNA (SEQ ID NO: 24)
catcatcaccatcaccatagcgatatcgagggccgcgcgcccatcagctcccactgcaggcttgacaagtccaacttccagc
agccctatatcaccaaccgcaccttcatgctggctaaggaggctagcttggctgataacaacacagacgttcgtctcattggggagaaac
tgttccacggagtcagtatgagtgagcgctgctatctgatgaagcaggtgctgaacttcacccttgaagaagtgctgttccctcaatctgat
aggttccagccttatatgcaggaggtggtgcccttcctggccaggctcagcaacaggctaagcacatgtcatattgaaggtgatgacctg
catatccagaggaatgtgcaaaagctgaaggacacagtgaaaaagcttggagagagtggagagatcaaagcaattggagaactggatt
tgctgtttatgtctctgagaaatgcctgcatttga
IL-22 mutant (SEQ ID NO: 25)
PISSHCRLDKSNFQQPAITNRTFMLAKEASLADQNTDVRLIGEKLFHGVSMSER
CYLMKQVLQFTLEEVFKNYQTPSHDYMSSVANFLARLSNRLSTCHIEGDDLHIQRNVQ
KLKDTVKKLGESGEIKAIGELDLLFMSLRNACI
IL-22 mutant cDNA (SEQ ID NO: 26)
atcaccaccatcaccactccgatattgaaggccgagctcccatttcgtcgcattgccgcctggacaagagcaactttcaacag
cctgcaatcacgaatcggacgtttatgttggcgaaggaagcaagcctggccgatcagaacacagatgtccgcctgatcggagagaagc
tcttccatggcgtgagcatgtccgagaggtgctacttgatgaaacaagtgttgcagttcactttggaagaagttttcaagaactatcagacc
ccctcccacgactacatgtcgtcggtggccaactttttggcgcgtctgagtaaccgactgagtacctgtcacatcgagggtgacgatctgc
acattcagcggaatgtacagaagctgaaagacaccgtcaagaaactcggtgagagcggagagatcaaggctataggcgagctggatc
tgctgttcatgtccctccgcaatgcctgcatataac
IL-22R1(d1)-IL-20R1(d2)hybrid (SEQ ID NO: 27)
AAAHAPEDPSDLLQHVKFQSSNMENILTWDSGPEGTPDTVYSIEYKTYGERDW
VAKKGCQRITRKSCNLSAETSNYTELYYARVTAVSAGGRSATKMTDRFYPFLETQIGPP
EVALTTDEKSISVVLTAPEKWKRNPEDLPVSMQQIYSNLKYNVSVLNTKSNRTWSQCV
TNHTLVLTWLEPNTLYCVHVESFVPGPPRRAQPSEKQCARTLKDQSSEFKAKGS
IL-22R1(d1)-IL-20R1(d2)hybrid cDNA (SEQ ID NO: 28)
gccgcggcacatgcgccagaggacccatcggatctgttgcagcacgttaagtttcagtcgagcaacatggagaacatactga
catgggattcgggtccggagggcacgccggacacagtgtatagtattgagtacaagacgtatggcgagagggactgggttgcgaaaa
agggctgccagcgaatcacccgaaaaagttgcaatctgtccgccgaaacctccaattacaccgagttgtactacgctcgcgtcactgctg
tcagcgccggaggccggtccgccacgaaaatgacggaccgcttctatccgtttttggagactcagatcggtcctcccgaggttgcactg
accacagatgagaagtccatttcggtcgtgctcactgccccagagaagtggaagcgcaaccccgaagatttgccggtgtcgatgcagc
agatctacagcaatctgaagtataacgtgagcgtactcaacaccaagtcgaaccggacatggtcccaatgtgtgaccaatcataccctcg
tgctgacatggctggagcccaacacactctactgcgtgcacgtggaaagtttcgtacccggacctccccgtcgcgcccaacccagcga
aaagcaatgcgcccgtacgctgaaggatcagagtagcgagttcaaggcaaaaggatccgccgcggcacatgcgccagaggacccat
cggatctgttgcagcacgttaagtttcagtcgagcaacatggagaacatactgacatgggattcgggtccggagggcacgccggacac
agtgtatagtattgagtacaagacgtatggcgagagggactgggttgcgaaaaagggctgccagcgaatcacccgaaaaagttgcaat
ctgtccgccgaaacctccaattacaccgagttgtactacgctcgcgtcactgctgtcagcgccggaggccggtccgccacgaaaatgac
ggaccgcttctatccgtttttggagactcagatcggtcctcccgaggttgcactgaccacagatgagaagtccatttcggtcgtgctcactg
ccccagagaagtggaagcgcaaccccgaagatttgccggtgtcgatgcagcagatctacagcaatctgaagtataacgtgagcgtactc
aacaccaagtcgaaccggacatggtcccaatgtgtgaccaatcataccctcgtgctgacatggctggagcccaacacactctactgcgt
gcacgtggaaagtttcgtacccggacctccccgtcgcgcccaacccagcgaaaagcaatgcgcccgtacgctgaaggatcagagtag
cgagttcaaggcaaaaggatcc
IL-20R1(D1)/IL-22R1(D2) hybrid (SEQ ID NO: 29)
AVPCVSGGLPKPANITFLSSNFENVLQWTPPEGLQGVKVTYTVQYFIYGQKKW
LNKSECRNINRTYCDLSAETSDLEHQYYAKVKAIWGTKCSKWAESGRFSSLQHTTLKP
PDVTCISKVRSIQMIVHPTPTPIRAGDGHRLTLEDIFHDLFYHLELQVNRTYQMHLGGK
QREYEFFGLTPDTEFLGTIMICVPTWAKESAPYMCRVKTLPDRT
IL-20R1(D1)/IL-22R1(D2) hybrid cDNA (SEQ ID NO: 30)
gccgcggccgtgccctgtgtttcgggcggtctgcccaagcctgctaatatcacgtttttgtccagcaatttcgagaacgtgctcc
agtggacgccaccggaaggattgcagggcgtgaaggtcacatataccgtgcagtacttcatatacggacagaaaaagtggttgaacaa
gagcgagtgtcgtaatatcaacaggacctactgcgatctgtcggcagaaacctcggatctggagcatcagtactacgctaaggtcaagg
ccatctggggcaccaagtgcagcaagtgggccgaaagtggacgattctccagcctgcaacacacaaccctgaagccgccagacgtca
catgcatctccaaagtgcgcagtatccagatgatcgtgcatcccacacccaccccgattcgtgcaggcgacggtcaccggctgactctg
gaggacatttttcacgatctgttctaccatctggagttgcaagtaaaccgcacttatcagatgcacctcggtggcaaacaacgcgagtatga
gttctttggcctgacaccagataccgaatttctcggcaccataatgatttgcgtacctacttgggccaaagagagtgccccctatatgtgca
gggttaagacattgccggaccgcacgtcgggatcc
IL-22R1/IL-20R1-FCk Hybrid (SEQ ID NO: 31)
HAPEDPSDLLQHVKFQSSNMENILTWDSGPEGTPDTVYSIEYKTYGERDWVAK
KGCQRITRKSCNLSAETSNYTELYYARVTAVSAGGRSATKMTDRFYPFLETQIGPPEVAL
TTDEKSISVVLTAPEKWKRNPEDLPVSMQQIYSNLKYNVSVLNTKSNRTWSQCVTNHT
LVLTWLEPNTLYCVHVESFVPGPPRRAQPSEKQCARTLKDQSSEFKAKGSGGGGPSVFI
FPPKIKDVLMISLSPIVTCVVVDVSEDDPDVQISWFVNNVEVHTAQTQTHREDYNSTLR
VVSALPIQHQDWMSGKEFKCKVNNKDLPAPIERTISKPKGSVRAPQVYVLPPPCEEMT
KKQVTLYCMVTDFMPEDIYVEWTNNGKTELNYKNTEPVLDSDGSYFMYSKLRVEKK
NWVERNSYSCSVVHEGLHNHHTTKSFSRTPGKGLEASGTGHHHHHH*
IL-22R1/IL-20R1-FCk Hybrid cDNA (SEQ ID NO: 32)
catgcgccagaggacccatcggatctgttgcagcacgttaagtttcagtcgagcaacatggagaacatactgacatgggattc
gggtccggagggcacgccggacacagtgtatagtattgagtacaagacgtatggcgagagggactgggttgcgaaaaagggctgcca
gcgaatcacccgaaaaagttgcaatctgtccgccgaaacctccaattacaccgagttgtactacgctcgcgtcactgctgtcagcgccgg
aggccggtccgccacgaaaatgacggaccgcttctatccgtttttggagactcagatcggtcctcccgaggttgcactgaccacagatga
gaagtccatttcggtcgtgctcactgccccagagaagtggaagcgcaaccccgaagatttgccggtgtcgatgcagcagatctacagca
atctgaagtataacgtgagcgtactcaacaccaagtcgaaccggacatggtcccaatgtgtgaccaatcataccctcgtgctgacatggct
ggagcccaacacactctactgcgtgcacgtggaaagtttcgtacccggacctccccgtcgcgcccaacccagcgaaaagcaatgcgc
ccgtacgctgaaggatcagagtagcgagttcaaggcaaaaggatccggaggaggtggtccatccgtcttcatcttccctccaaagatca
aggatgtactcatgatctccctgagccccatagtcacatgtgtggtggtggatgtgagcgaggatgacccagatgtccagatcagctggtt
tgtgaacaacgtggaagtacacacagctcagacacaaacccatagagaggattacaacagtactctccgggtggtcagtgccctcccca
tccagcaccaggactggatgagtggcaaggagttcaaatgcaaggtcaacaacaaagacctcccagcgcccatcgagagaaccatct
caaaacccaaagggtcagtaagagctccacaggtatatgtcttgcctccaccatgcgaagagatgactaagaaacaggtcactctgtact
gcatggtcacagacttcatgcctgaagacatttacgtggagtggaccaacaacgggaaaacagagctaaactacaagaacactgaacc
agtcctggactctgatggttcttacttcatgtacagcaagctgagagtggaaaagaagaactgggtggaaagaaatagctactcctgttca
gtggtccacgagggtctgcacaatcaccacacgactaagagcttctcccggactccgggtaaaggtctcgaggctagcggaaccggtc
atcatcaccatcaccattga
IL-20R1/IL-22R1-FCk Hybrid (SEQ ID NO: 33)
VPCVSGGLPKPANITFLSSNFENVLQWTPPEGLQGVKVTYTVQYFIYGQKKWL
NKSECRNINRTYCDLSAETSDLEHQYYAKVKAIWGTKCSKWAESGRFSSLQHTTLKPP
DVTCISKVRSIQMIVHPTPTPIRAGDGHRLTLEDIFHDLFYHLELQVNRTYQMHLGGKQ
REYEFFGLTPDTEFLGTIMICVPTWAKESAPYMCRVKTLPDRTSGSGGGGPSVFIFPPKI
KDVLMISLSPIVTCVVVDVSEDDPDVQISWFVNNVEVHTAQTQTHREDYNSTLRVVSA
LPIQHQDWMSGKEFKCKVNNKDLPAPIERTISKPKGSVRAPQVYVLPPPCEEMTKKQV
TLYCMVTDFMPEDIYVEWTNNGKTELNYKNTEPVLDSDGSYFMYSKLRVEKKNWVE
RNSYSCSVVHEGLHNHHTTKSFSRTPGKGLEASGGLNDIFEAQKIEWHEGTGHHHHH
H
IL-20R1/IL-22R1-FCk Hybrid cDNA (SEQ ID NO: 34)
gtgccctgtgtttcgggcggtctgcccaagcctgctaatatcacgtttttgtccagcaatttcgagaacgtgctccagtggacgc
caccggaaggattgcagggcgtgaaggtcacatataccgtgcagtacttcatatacggacagaaaaagtggttgaacaagagcgagtgt
cgtaatatcaacaggacctactgcgatctgtcggcagaaacctcggatctggagcatcagtactacgctaaggtcaaggccatctgggg
caccaagtgcagcaagtgggccgaaagtggacgattctccagcctgcaacacacaaccctgaagccgccagacgtcacatgcatctcc
aaagtgcgcagtatccagatgatcgtgcatcccacacccaccccgattcgtgcaggcgacggtcaccggctgactctggaggacattttt
cacgatctgttctaccatctggagttgcaagtaaaccgcacttatcagatgcacctcggtggcaaacaacgcgagtatgagttctttggcct
gacaccagataccgaatttctcggcaccataatgatttgcgtacctacttgggccaaagagagtgccccctatatgtgcagggttaagaca
ttgccggaccgcacgtcgggatccggaggaggtggtccatccgtcttcatcttccctccaaagatcaaggatgtactcatgatctccctga
gccccatagtcacatgtgtggtggtggatgtgagcgaggatgacccagatgtccagatcagctggtttgtgaacaacgtggaagtacac
acagctcagacacaaacccatagagaggattacaacagtactctccgggtggtcagtgccctccccatccagcaccaggactggatga
gtggcaaggagttcaaatgcaaggtcaacaacaaagacctcccagcgcccatcgagagaaccatctcaaaacccaaagggtcagtaa
gagctccacaggtatatgtcttgcctccaccatgcgaagagatgactaagaaacaggtcactctgtactgcatggtcacagacttcatgcc
tgaagacatttacgtggagtggaccaacaacgggaaaacagagctaaactacaagaacactgaaccagtcctggactctgatggttctta
cttcatgtacagcaagctgagagtggaaaagaagaactgggtggaaagaaatagctactcctgttcagtggtccacgagggtctgcaca
atcaccacacgactaagagcttctcccggactccgggtaaaggtctcgaggctagcggaggactcaacgacatcttcgaagcacaaaa
aatcgaatggcacgaaggtaccggtcatcatcaccatcaccattga
SEQ ID NO: 35
GLKTLNLGSC
SEQ ID NO: 36
ASSMKTLNLGSC
SEQ ID NO: 37
CSSMKTLNLG SC
SEQ ID NO: 38
ASSMKTLNLGSCVIATNLQEIRNGFSEIRGSVQAKDGNIDIRILRRTESLQDTKPANRCC
LLRHLLRLYLDRVFKNYQTPDHYTLRKISSLANSFLTIKKDLRLCHAHMTCHCGEEAM
KKYSQILSHFEKLEPQAAVVKALGELDILLQWMEETE
SEQ ID NO: 39
CSSMKTLNLGSCVIATNLCEIRNGFSEIRGSVQAKDGNIDIRILRRTESLQDTKPANRCC
LLRHLLRLYLDRVFKNYQTPDHYTLRKISSLANSFLTIKKDLRLCHAHMTCHCGEEAM
KKYSQILSHFEKLEPQAAVVKALGELDILLQWMEETE
SEQ ID NO: 40
HCRLDKSNFQQPYITNRTFMLAKEASLADQNTDVRLIGEKLFHGVSMSERCYLMKQV
LQFTLEEVLFPQSDRFQPYMQEVVPFLARLSNRLSTCHIEGDDLHIQRNVQKLKDTVK
KLGESGEIKAIGELDLLFMSLRNACI
SEQ ID NO: 41
HCRLDKSNFQQPAITNRTFMLAKEASLADQNTDVRLIGEKLFHGVSMSERCYLMKQV
LQFTLEEVFKNYQTPSHDYMSSVANFLARLSNRLSTCHIEGDDLHIQRNVQKLKDTVK
KLGESGEIKAIGELDLLFMSLRNACI
SEQ ID NO: 42
IEGRLESRGPFEGKPIPNPLLGLDSTRTGHHHHHH
SEQ ID NO: 43
IEGRVESRGPFEGKPIPNPLLGLDSTRTGHHHHHH
SEQ ID NO: 44
HHHHHHSDIEGRA
SEQ ID NO: 45
HMNHGLRRCLISTDLQEIRNGFSEIRGSVQAKDGNIDIRILRRTESLQDTKPANRCCLL
RHLLRLYLDRVFKNYQTPDHYTLRKISSLANSFLTIKKDLRLCHAHMTCHCGEEAMK
KYSQILSHFEKLEPQAAVVKALGELDILLQWMEEHEVMFSA

1. A composition comprising one or more agents that inhibit or reduce the activity of IL-20.

2. The composition of claim 1, wherein the agent selectively binds an IL-20R1/IL-20R2 receptor complex.

3. (canceled)

4. The composition of claim 1, wherein the agent comprises SEQ ID NO:3 comprising one or more mutations.

5. (canceled)

6. (canceled)

7. The composition of claim 1, wherein the agent comprises SEQ ID NO:19.

8. The composition of claim 1, wherein the agent comprises SEQ ID NO:20.

9. (canceled)

10. The composition of claim 1, wherein the agent comprises SEQ ID NO:21.

11. (canceled)

12. (canceled)

13. (canceled)

14. The composition of claim 1, wherein the agent selectively binds an IL-22R1/IL-20R2 receptor complex.

15. (canceled)

16. The composition of claim 14, wherein the agent comprises SEQ ID NO:23 comprising one or more mutations.

17. (canceled)

18. (canceled)

19. (canceled)

20. (canceled)

21. The composition of claim 14, wherein the agent comprises SEQ ID NO:25.

22. The composition of claim 1, wherein the agent further inhibits or reduces the activity of IL-24.

23. The composition of claim 1, wherein the agent further inhibits or reduces the activity of IL-24 and wherein the agent prevents or reduces IL-24 binding to the IL-22R1/IL-20R2 complex.

24. The composition of claim 14, wherein the agent further inhibits or reduces the activity of IL-24 and wherein the agent prevents or reduces IL-24 binding to the IL-20R1/IL-20R2 complex.

25. The composition of claim 1, wherein the agent selectively binds IL-20 and IL-24.

26. The composition of claim 1, wherein the composition comprises SEQ ID NO: 27.

27. (canceled)

28. (canceled)

29. (canceled)

30. (canceled)

31. (canceled)

32. (canceled)

33. (canceled)

34. (canceled)

35. A method of treating or preventing an inflammatory or autoimmune disease in a subject comprising administering an effective amount of the composition of claim 1 to the subject, wherein administering the effective amount of the composition treats or prevents the inflammatory or autoimmune disease in the subject.

36. (canceled)

37. (canceled)

38. A method for screening for agents that selectively bind IL-20R1/IL-20R2 receptor complex comprising contacting an agent to be screened with the IL-20R1/IL-20R2 receptor and determining whether the agent binds the IL-20R1/IL-20R2 complex.

39. The method of claim 38, wherein binding indicates the agent inhibits or reduces the activity of IL-20.

40. (canceled)

41. (canceled)

42. (canceled)

43. (canceled)

44. (canceled)

45. (canceled)

46. (canceled)

47. (canceled)

48. A method for screening for agents that selectively bind IL-22R1/IL-20R2 receptor complex comprising contacting an agent to be screened with the IL-22R1/IL-20R2 receptor and determining whether the agent binds the IL-22R1/IL-20R2 complex.

49. The method of claim 48, wherein binding indicates the agent inhibits or reduces the activity of IL-20.

50. (canceled)

51. (canceled)

52. (canceled)

53. (canceled)

54. (canceled)

55. A kit comprising the composition of claim 1 and instructions for use.


SEQUENCE LISTING (45 sequences)

SEQ ID NO: 1 - protein, 161 aa, Homo sapiens
MASVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 2 - DNA, 486 bp, Homo sapiens
atggcctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaagaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagcttga

SEQ ID NO: 3 - protein, 153 aa, Homo sapiens
MLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 4 - DNA, 462 bp, Homo sapiens
atgctgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaatag

SEQ ID NO: 5 - protein, 216 aa, Homo sapiens
VPCVSGGLPKPANITFLSINMKNVLQWTPPEGLQGVKVTYTVQYFIYGQKKWLNKSECRNINRTYCDLSAETSDYEHQYYAKVKAIWGTKCSKWAESGRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSS

SEQ ID NO: 6 - DNA, 756 bp, Homo sapiens
gttccctgtgtctctggtggtttgcctaaacctgcaaacatcaccttcttatccatcaacatgaagaatgtcctacaatggactccaccagagggtcttcaaggagttaaagttacttacactgtgcagtatttcatatatgggcaaaagaaatggctgaataaatcagaatgcagaaatatcaatagaacctactgtgatctttctgctgaaacttctgactacgaacaccagtattatgccaaagttaaggccatttggggaacaaagtgttccaaatgggctgaaagtggacggttctatccttttttagaaacacaaattggcccaccagaggtggcactgactacagatgagaagtccatttctgttgtcctgacagctccagagaagtggaagagaaatccagaagaccttcctgtttccatgcaacaaatatactccaatctgaagtataacgtgtctgtgttgaatactaaatcaaacagaacgtggtcccagtgtgtgaccaaccacacgctggtgctcacctggctggagccgaacactctttactgcgtacacgtggagtccttcgtcccagggccccctcgccgtgctcagccttctgagaagcagtgtgccaggactttgaaagatcaatcatcaattgaaggtagactcgagtctagagggcccttcgaaggtaagcctatccctaaccctctcctcggtctcgattctacgcgtaccggtcatcatcaccatcaccattga

SEQ ID NO: 7 - protein, 201 aa, Homo sapiens
DEVAILPAPQQLSVLSTNMKHLLMWSPVIAPGETVYYSVEYQGEYESLYTSHIWIPSSWCSLTEGPECDVTDDITATVPYNLRVRATLGSQTSAWSILKHPFNRQSTILTRPGMEITKDGFHLVIELEDLGPQFEFLVAYWRREPGAEEHVKMVRSGGIPVHLETMEPGAAYCVKAQTFVKAIGRYSAFSQTECVEVQGEA

SEQ ID NO: 8 - DNA, 710 bp, Homo sapiens
gatgaagtggccattctgcctgcccctcagcagctctctgtactctcaaccaacatgaagcatctcttgatgtggagcccagtgatcgcgcctggagaaacagtgtactattctgtcgaataccagggggagtacgagagcctgtacacgagccacatctggatccccagcagctggtgctcactcactgaaggtcctgagtgtgatgtcactgatgacatcacggccactgtgccatacaaccttcgtgtcagggccacattgggctcacagacctcagcctggagcatcctgaagcatccctttaatagaaactcaaccatccttacccgacctgggatggagatcaccaaagatggcttccacctggttattgagctggaggacctggggccccagtttgagttccttgtggcctactggaggagggagcctggtgccgaggaacatgtcaaaatggtgaggagtgggggtattccagtgcacctagaaaccatggagccaggggctgcatactgtgtgaaggcccagacattcgtgaaggccattgggaggtacagcgccttcagccagacagaatgtgtggaggtgcaaggagaggccattgaaggtagactcgagtctagagggcccttcgaaggtaagcctatccctaaccctctcctcggtctcgattctacgcgtaccggtcatcatcaccatcaccattg

SEQ ID NO: 9 - protein, 211 aa, Homo sapiens
HAPEDPSDLLQHVKFQSSNFENILTWDSGPEGTPDTVYSIEYKTYGERDWVAKKGCQRITRKSCDLTVETGDLQELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRT

SEQ ID NO: 10 - DNA, 741 bp, Homo sapiens
cacgcccctgaggacccctcggatctgctccagcacgtgaaattccagtccagcaactttgaaaacatcctgacgtgggacagcgggccagagggcaccccagacacggtctacagcatcgagtataagacgtacggagagagggactgggtggcaaagaagggctgtcagcggatcacccggaagtcctgcgacctgacggtggagacgggcgacctccaggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacaattgaaggtagagtcgagtctagagggcccttcgaaggtaagcctatccctaaccctctcctcggtctcgattctacgcgtaccggtcatcatcaccatcaccattga

SEQ ID NO: 11 - protein, 467 aa, Artificial sequence
  Synthetic construct
VPCVSGGLPKPANITFLSINMKNVLQWTPPEGLQGVKVTYTVQYFIYGQKKWLNKSECRNINRTYCDLSAETSDYEHQYYAKVKAIWGTKCSKWAESGRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKAKGSGGGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPCEEMTKKQVTLYCMVTDFMPEDIYVEWTNNGKTELNYKNTEPVLDSDGSYFMYSKLRVEKKNWVERNSYSCSVVHEGLHNHHTTKSFSRTPGKGLEASGGLNDIFEAQKIEWHEGTGHHHHHH

SEQ ID NO: 12 - DNA, 1404 bp, Artificial sequence
  Synthetic construct
gttccctgtgtctctggtggtttgcctaaacctgcaaacatcaccttcttatccatcaacatgaagaatgtcctacaatggactccaccagagggtcttcaaggagttaaagttacttacactgtgcagtatttcatatatgggcaaaagaaatggctgaataaatcagaatgcagaaatatcaatagaacctactgtgatctttctgctgaaacttctgactacgaacaccagtattatgccaaagttaaggccatttggggaacaaagtgttccaaatgggctgaaagtggacggttctatccttttttagaaacacaaattggcccaccagaggtggcactgactacagatgagaagtccatttctgttgtcctgacagctccagagaagtggaagagaaatccagaagaccttcctgtttccatgcaacaaatatactccaatctgaagtataacgtgtctgtgttgaatactaaatcaaacagaacgtggtcccagtgtgtgaccaaccacacgctggtgctcacctggctggagccgaacactctttactgcgtacacgtggagtccttcgtcccagggccccctcgccgtgctcagccttctgagaagcagtgtgccaggactttgaaagatcaatcatcagagtttaaagccaaaggatccggaggaggtggtccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccatgcgaagagatgactaagaaacaggtcactctgtactgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtctcgaggctagcggaggactcaacgacatcttcgaagcacaaaaaatcgaatggcacgaaggtaccggtcatcatcaccatcaccattga

SEQ ID NO: 13 - protein, 462 aa, Artificial sequence
  Synthetic construct
AHAPEDPSDLLQHVKFQSSNFENILTWDSGPEGTPDTVYSIEYKTYGERDWVAKKGCQRITRKSCDLTVETGDLQELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTYSGSGGGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPCEEMTKKQVTLYCMVTDFMPEDIYVEWTNNGKTELNYKNTEPVLDSDGSYFMYSKLRVEKKNWVERNSYSCSVVHEGLHNHHTTKSFSRTPGKGLEASGGLNDIFEAQKIEWHEGTGHHHHHH

SEQ ID NO: 14 - DNA, 1389 bp, Artificial sequence
  Synthetic construct
gctcacgcccctgaggacccctcggatctgctccagcacgtgaaattccagtccagcaactttgaaaacatcctgacgtgggacagcgggccagagggcaccccagacacggtctacagcatcgagtataagacgtacggagagagggactgggtggcaaagaagggctgtcagcggatcacccggaagtcctgcgacctgacggtggagacgggcgacctccaggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatggacctactccggatccggaggaggtggtccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccatgcgaagagatgactaagaaacaggtcactctgtactgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtctcgaggctagcggaggactcaacgacatcttcgaagcacaaaaaatcgaatggcacgaaggtaccggtcatcatcaccatcaccattga

SEQ ID NO: 15 - protein, 435 aa, Artificial sequence
  Synthetic construct
DEVAILPAPQQLSVLSTNMKHLLMWSPVIAPGETVYYSVEYQGEYESLYTSHIWIPSSWCSLTEGPECDVTDDITATVPYNLRVRATLGSQTSAWSILKHPFNRQSTILTRPGMEITKDGFHLVIELEDLGPQFEFLVAYWRREPGAEEHVKMVRSGGIPVHLETMEPGAAYCVKAQTFVKAIGRYSAFSQTECVEVQGEAGSGGGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMTSKLRVEKKNWVERNSYSCSVVHEGLHNHHTTKSFGGCGTPASGGLNDIFEAQKIEWHE

SEQ ID NO: 16 - DNA, 1308 bp, Artificial sequence
  Synthetic construct
gatgaagtggccattctgcctgcccctcagcagctctctgtactctcaaccaacatgaagcatctcttgatgtggagcccagtgatcgcgcctggagaaacagtgtactattctgtcgaataccagggggagtacgagagcctgtacacgagccacatctggattcccagcagctggtgctcactcactgaaggtcctgagtgtgatgtcactgatgacatcacggccactgtgccatacaaccttcgtgtcagggccacattgggctcacagacctcagcctggagcatcctgaagcatccctttaatagacagtcaaccatccttacccgacctgggatggagatcaccaaagatggcttccacctggttattgagctggaggacctggggccccagtttgagttccttgtggcctactggaggagggagcctggtgccgaggaacatgtcaaaatggtgaggagtgggggtattccagtgcacctagaaaccatggagccaggggctgcatactgtgtgaaggcccagacattcgtgaaggccattgggaggtacagcgccttcagccagacagaatgtgtggaggtgcaaggagaggccggatccggaggaggtggtccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgaccagcaagctgagagtggaaaagaagaactgggtggaaagaaatagctactcctgttcagtggtccacgagggtctgcacaatcaccacacgactaagagcttcggaggttgcggaacaccagctagcggaggactcaacgacatcttcgaagcacaaaaaatcgaatggcacgaatga

SEQ ID NO: 17 - protein, 158 aa, Homo sapiens
AQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 18 - DNA, 477 bp, Homo sapiens
gcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcattcaaacagttggacgtagaagcagctctgaccaaagcccttggggaagtggacattcttctgacctggatgcagaaattctacaagctctga

SEQ ID NO: 19 - protein, 154 aa, Artificial sequence
  Synthetic construct
SSMKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 20 - protein, 155 aa, Homo sapiens
CSSMKTLNLGSCVIATNLCEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 21 - protein, 156 aa, Artificial sequence
  Synthetic construct
MNHGLRRCLISTDLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEEHEVMFSA

SEQ ID NO: 22 - DNA, 471 bp, Artificial sequence
  synthetic construct
atgaaccacggtctcaggagatgtctgatttccacagaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagcatgaagtaatgttctcagcttag

SEQ ID NO: 23 - protein, 145 aa, Homo sapiens
PISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 24 - DNA, 477 bp, Homo sapiens
catcatcaccatcaccatagcgatatcgagggccgcgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttga

SEQ ID NO: 25 - protein, 145 aa, Artificial sequence
  Synthetic construct
PISSHCRLDKSNFQQPAITNRTFMLAKEASLADQNTDVRLIGEKLFHGVSMSERCYLMKQVLQFTLEEVFKNYQTPSHDYMSSVANFLARLSNRLSTCHIEGDDLHIQRNVQKLKDTVKKLGESGEIKAIGELDLLFMSLRNACI

SEQ ID NO: 26 - DNA, 477 bp, Artificial sequence
  Synthetic construct
atcaccaccatcaccactccgatattgaaggccgagctcccatttcgtcgcattgccgcctggacaagagcaactttcaacagcctgcaatcacgaatcggacgtttatgttggcgaaggaagcaagcctggccgatcagaacacagatgtccgcctgatcggagagaagctcttccatggcgtgagcatgtccgagaggtgctacttgatgaaacaagtgttgcagttcactttggaagaagttttcaagaactatcagaccccctcccacgactacatgtcgtcggtggccaactttttggcgcgtctgagtaaccgactgagtacctgtcacatcgagggtgacgatctgcacattcagcggaatgtacagaagctgaaagacaccgtcaagaaactcggtgagagcggagagatcaaggctataggcgagctggatctgctgttcatgtccctccgcaatgcctgcatataac

SEQ ID NO: 27 - protein, 224 aa, artificial sequence
  Synthetic construct
AAAHAPEDPSDLLQHVKFQSSNMENILTWDSGPEGTPDTVYSIEYKTYGERDWVAKKGCQRITRKSCNLSAETSNYTELYYARVTAVSAGGRSATKMTDRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKAKGS

SEQ ID NO: 28 - DNA, 1344 bp, Artificial sequence
  Synthetic construct
gccgcggcacatgcgccagaggacccatcggatctgttgcagcacgttaagtttcagtcgagcaacatggagaacatactgacatgggattcgggtccggagggcacgccggacacagtgtatagtattgagtacaagacgtatggcgagagggactgggttgcgaaaaagggctgccagcgaatcacccgaaaaagttgcaatctgtccgccgaaacctccaattacaccgagttgtactacgctcgcgtcactgctgtcagcgccggaggccggtccgccacgaaaatgacggaccgcttctatccgtttttggagactcagatcggtcctcccgaggttgcactgaccacagatgagaagtccatttcggtcgtgctcactgccccagagaagtggaagcgcaaccccgaagatttgccggtgtcgatgcagcagatctacagcaatctgaagtataacgtgagcgtactcaacaccaagtcgaaccggacatggtcccaatgtgtgaccaatcataccctcgtgctgacatggctggagcccaacacactctactgcgtgcacgtggaaagtttcgtacccggacctccccgtcgcgcccaacccagcgaaaagcaatgcgcccgtacgctgaaggatcagagtagcgagttcaaggcaaaaggatccgccgcggcacatgcgccagaggacccatcggatctgttgcagcacgttaagtttcagtcgagcaacatggagaacatactgacatgggattcgggtccggagggcacgccggacacagtgtatagtattgagtacaagacgtatggcgagagggactgggttgcgaaaaagggctgccagcgaatcacccgaaaaagttgcaatctgtccgccgaaacctccaattacaccgagttgtactacgctcgcgtcactgctgtcagcgccggaggccggtccgccacgaaaatgacggaccgcttctatccgtttttggagactcagatcggtcctcccgaggttgcactgaccacagatgagaagtccatttcggtcgtgctcactgccccagagaagtggaagcgcaaccccgaagatttgccggtgtcgatgcagcagatctacagcaatctgaagtataacgtgagcgtactcaacaccaagtcgaaccggacatggtcccaatgtgtgaccaatcataccctcgtgctgacatggctggagcccaacacactctactgcgtgcacgtggaaagtttcgtacccggacctccccgtcgcgcccaacccagcgaaaagcaatgcgcccgtacgctgaaggatcagagtagcgagttcaaggcaaaaggatcc

SEQ ID NO: 29 - protein, 214 aa, Artificial sequence
  Synthetic construct
AVPCVSGGLPKPANITFLSSNFENVLQWTPPEGLQGVKVTYTVQYFIYGQKKWLNKSECRNINRTYCDLSAETSDLEHQYYAKVKAIWGTKCSKWAESGRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRT

SEQ ID NO: 30 - DNA, 657 bp, Artificial sequence
  Synthetic construct
gccgcggccgtgccctgtgtttcgggcggtctgcccaagcctgctaatatcacgtttttgtccagcaatttcgagaacgtgctccagtggacgccaccggaaggattgcagggcgtgaaggtcacatataccgtgcagtacttcatatacggacagaaaaagtggttgaacaagagcgagtgtcgtaatatcaacaggacctactgcgatctgtcggcagaaacctcggatctggagcatcagtactacgctaaggtcaaggccatctggggcaccaagtgcagcaagtgggccgaaagtggacgattctccagcctgcaacacacaaccctgaagccgccagacgtcacatgcatctccaaagtgcgcagtatccagatgatcgtgcatcccacacccaccccgattcgtgcaggcgacggtcaccggctgactctggaggacatttttcacgatctgttctaccatctggagttgcaagtaaaccgcacttatcagatgcacctcggtggcaaacaacgcgagtatgagttctttggcctgacaccagataccgaatttctcggcaccataatgatttgcgtacctacttgggccaaagagagtgccccctatatgtgcagggttaagacattgccggaccgcacgtcgggatcc

SEQ ID NO: 31 - protein, 449 aa, Artificial sequence
  Synthetic construct
HAPEDPSDLLQHVKFQSSNMENILTWDSGPEGTPDTVYSIEYKTYGERDWVAKKGCQRITRKSCNLSAETSNYTELYYARVTAVSAGGRSATKMTDRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKAKGSGGGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPCEEMTKKQVTLYCMVTDFMPEDIYVEWTNNGKTELNYKNTEPVLDSDGSYFMYSKLRVEKKNWVERNSYSCSVVHEGLHNHHTTKSFSRTPGKGLEASGTGHHHHHH

SEQ ID NO: 32 - DNA, 1350 bp, Artificial sequence
  Synthetic construct
catgcgccagaggacccatcggatctgttgcagcacgttaagtttcagtcgagcaacatggagaacatactgacatgggattcgggtccggagggcacgccggacacagtgtatagtattgagtacaagacgtatggcgagagggactgggttgcgaaaaagggctgccagcgaatcacccgaaaaagttgcaatctgtccgccgaaacctccaattacaccgagttgtactacgctcgcgtcactgctgtcagcgccggaggccggtccgccacgaaaatgacggaccgcttctatccgtttttggagactcagatcggtcctcccgaggttgcactgaccacagatgagaagtccatttcggtcgtgctcactgccccagagaagtggaagcgcaaccccgaagatttgccggtgtcgatgcagcagatctacagcaatctgaagtataacgtgagcgtactcaacaccaagtcgaaccggacatggtcccaatgtgtgaccaatcataccctcgtgctgacatggctggagcccaacacactctactgcgtgcacgtggaaagtttcgtacccggacctccccgtcgcgcccaacccagcgaaaagcaatgcgcccgtacgctgaaggatcagagtagcgagttcaaggcaaaaggatccggaggaggtggtccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccatgcgaagagatgactaagaaacaggtcactctgtactgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtctcgaggctagcggaaccggtcatcatcaccatcaccattga

SEQ ID NO: 33 - protein, 460 aa, Artificial sequence
  Synthetic construct
VPCVSGGLPKPANITFLSSNFENVLQWTPPEGLQGVKVTYTVQYFIYGQKKWLNKSECRNINRTYCDLSAETSDLEHQYYAKVKAIWGTKCSKWAESGRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTSGSGGGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPCEEMTKKQVTLYCMVTDFMPEDIYVEWTNNGKTELNYKNTEPVLDSDGSYFMYSKLRVEKKNWVERNSYSCSVVHEGLHNHHTTKSFSRTPGKGLEASGGLNDIFEAQKIEWHEGTGHHHHHH

SEQ ID NO: 34 - DNA, 1383 bp, Artificial sequence
  Synthetic construct
gtgccctgtgtttcgggcggtctgcccaagcctgctaatatcacgtttttgtccagcaatttcgagaacgtgctccagtggacgccaccggaaggattgcagggcgtgaaggtcacatataccgtgcagtacttcatatacggacagaaaaagtggttgaacaagagcgagtgtcgtaatatcaacaggacctactgcgatctgtcggcagaaacctcggatctggagcatcagtactacgctaaggtcaaggccatctggggcaccaagtgcagcaagtgggccgaaagtggacgattctccagcctgcaacacacaaccctgaagccgccagacgtcacatgcatctccaaagtgcgcagtatccagatgatcgtgcatcccacacccaccccgattcgtgcaggcgacggtcaccggctgactctggaggacatttttcacgatctgttctaccatctggagttgcaagtaaaccgcacttatcagatgcacctcggtggcaaacaacgcgagtatgagttctttggcctgacaccagataccgaatttctcggcaccataatgatttgcgtacctacttgggccaaagagagtgccccctatatgtgcagggttaagacattgccggaccgcacgtcgggatccggaggaggtggtccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccatgcgaagagatgactaagaaacaggtcactctgtactgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtctcgaggctagcggaggactcaacgacatcttcgaagcacaaaaaatcgaatggcacgaaggtaccggtcatcatcaccatcaccattga

SEQ ID NO: 35 - protein, 10 aa, Artificial sequence
  Synthetic construct
GLKTLNLGSC

SEQ ID NO: 36 - protein, 12 aa, Artificial sequence
  Synthetic construct
ASSMKTLNLGSC

SEQ ID NO: 37 - protein, 12 aa, Artificial sequence
  Synthetic construct
CSSMKTLNLGSC

SEQ ID NO: 38 - protein, 155 aa, Artificial sequence
  Synthetic construct
ASSMKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 39 - protein, 155 aa, Artificial sequence; identical to SEQ ID NO: 20
  Synthetic construct

SEQ ID NO: 40 - protein, 141 aa, Artificial sequence
  Synthetic construct
HCRLDKSNFQQPYITNRTFMLAKEASLADQNTDVRLIGEKLFHGVSMSERCYLMKQVLQFTLEEVLFPQSDRFQPYMQEVVPFLARLSNRLSTCHIEGDDLHIQRNVQKLKDTVKKLGESGEIKAIGELDLLFMSLRNACI

SEQ ID NO: 41 - protein, 141 aa, Artificial sequence
  Synthetic construct
HCRLDKSNFQQPAITNRTFMLAKEASLADQNTDVRLIGEKLFHGVSMSERCYLMKQVLQFTLEEVFKNYQTPSHDYMSSVANFLARLSNRLSTCHIEGDDLHIQRNVQKLKDTVKKLGESGEIKAIGELDLLFMSLRNACI

SEQ ID NO: 42 - protein, 35 aa, Artificial sequence
  synthetic construct
IEGRLESRGPFEGKPIPNPLLGLDSTRTGHHHHHH

SEQ ID NO: 43 - protein, 35 aa, Artificial sequence
  Synthetic construct
IEGRVESRGPFEGKPIPNPLLGLDSTRTGHHHHHH

SEQ ID NO: 44 - protein, 13 aa, Artificial sequence
  Synthetic construct
HHHHHHSDIEGRA

SEQ ID NO: 45 - protein, 157 aa, Artificial sequence
  synthetic construct
HMNHGLRRCLISTDLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEEHEVMFSA